(19) United States
(12) Patent Application Publication
(10) Pub. No.: US 2026/0183350 A1
(43) Pub. Date: Jul. 2, 2026

(54) ANTIBIOTIC-RESISTANT BACTERIA

(71) Applicant: Imperial College Innovations Limited, London Greater London (GB)

(72) Inventors: Julie MCDONALD, London Greater London (GB); Alexander YIP, London Greater London (GB); Olivia KING, London Greater London (GB)

(73) Assignee: Imperial College Innovations Limited, London Greater London (GB)

(21) Appl. No.: 19/129,141

(22) PCT Filed: Nov. 17, 2023
(86) PCT No.: PCT/GB2023/053021

§ 371 Date: May 12, 2025

(30) Foreign Application Priority Data
Nov. 18, 2022 (GB) 2217266.2

(51) Int. Cl. A61K 35/745; A61K 31/045; A61K 31/19; A61K 31/197; A61K 35/00; A61K 35/741

(52) U.S. Cl. CPC A61K 35/745; A61K 31/045; A61K 31/19; A61K 31/197; A61K 35/741; A61K 2035/115

(57) ABSTRACT

The present invention relates to antibiotic-resistant bacteria, and to the prevention or treatment of infections of drug-resistant pathogens, such as carbapenem-resistant Enterobacteriaceae (CRE) and/or vancomycin-resistant Enterococcus (VRE). In particular, the invention relates to the treatment or prevention of colonisation of CRE and/or VRE in the gastrointestinal (GI) tract, before they cause invasive infections, for example in the blood. The invention provides novel formulations, pharmaceutical compositions and methods of using them to prevent or treat CRE and/or VRE infections or intestinal colonisations thereof.

The present invention relates to antibiotic-resistant bacteria, and particularly, although not exclusively, to the prevention or treatment of infections of drug-resistant pathogens, such as carbapenem-resistant Enterobacteriaceae (CRE) and/or vancomycin-resistant Enterococcus (VRE). In particular, the invention relates to the treatment or prevention of colonisation of CRE and/or VRE in the gastrointestinal (GI) tract, before they cause invasive infections, for example in the blood. The invention provides novel formulations, pharmaceutical compositions and methods of using them to prevent or treat CRE and/or VRE infections or intestinal colonisations thereof.

Antibiotic resistance is a serious threat to human health, resulting in treatment failures, infection relapses, longer hospitalisations, and poor clinical outcomes. Treatment options are increasingly limited, less effective, and involve administration of more toxic antibiotics. There is an urgent need to develop new approaches to prevent and treat infections by antibiotic-resistant bacteria, in particular infections by extremely drug-resistant pathogens, such as carbapenem-resistant Enterobacteriaceae (CRE) and vancomycin-resistant Enterococcus (VRE). There has been a global increase in CRE and VRE over the past two decades, leading experts to list these “nightmare bacteria” as an urgent threat. CRE and VRE infections are associated with higher treatment failure rates and increased mortality rates (CRE sepsis mortality rates ˜50%). New approaches to prevent CRE and VRE infections are, therefore, needed.

The intestine is the primary colonisation site for CRE and VRE and serves as a reservoir of CRE and VRE that are responsible for difficult-to-treat invasive infections (e.g. bloodstream infections, recurrent urinary tract infections). Therefore, alternative methods to prevent the development of CRE and VRE invasive infections would be to prevent CRE and VRE from colonising the intestine or to decolonise patients with pre-existing CRE and VRE intestinal colonisation. The main risk factors for CRE and VRE intestinal colonisation and domination include antibiotic use (creating an ecological niche for CRE and VRE) and healthcare treatment (exposing patients to CRE and VRE). Studies have demonstrated CRE and VRE intestinal decolonisation and a reduction in CRE and VRE invasive infections following faecal microbiota transplantation (FMT), where faeces from a healthy donor are administered to a CRE and VRE colonised patient. CRE and VRE decolonisation rates range from 33-75% and can be increased if antibiotics are not administered post-FMT. However, FMT is not the ideal treatment for immunocompromised patients due to the risk of transmitting infections and frequent antibiotic exposure. FMT needs to be replaced with a safer, more effective treatment. Healthy gut microbiota exhibit colonisation resistance, where microbes compete for shared niches within the intestine. Pathogens must overcome colonisation resistance before they can colonise the intestine. It is well-established that broad spectrum antibiotics deplete commensal bacteria, resulting in a decrease in the diversity and biomass of bacterial taxa within the gut microbiota. Disruption of colonisation resistance by antibiotic-mediated killing of protective gut commensals significantly promotes the expansion of Enterobacteriaceae and Enterococcus within the gut. FMT has been proposed to restore colonisation resistance against CRE and VRE by reintroducing the gut commensals that mediate this colonisation resistance. The mechanisms of colonisation resistance that healthy microbiota use to protect against CRE and VRE intestinal colonisation are not fully described.

Pathogens must have access to nutrients that can support their growth to successfully colonise the intestine. However, nutrients are limited within the intestine and bacteria with similar or “overlapping” nutrient utilisation abilities will compete for similar niches. Ecological niches within the gut are defined by the availability of different nutrients. Different bacterial species will have different nutrient utilisation abilities, and the diversity and concentration of the available nutrients will impact their growth. Although bacteria may be able to utilise a variety of nutrients, they generally have a prioritised utilisation of nutrients that varies between different bacterial species. Polysaccharides, proteins, and mucins (heavily glycosylated proteins) are major food sources for the gut microbiota in the large intestine. Primary degraders can liberate simple compounds from these complex substrates, including monosaccharides, disaccharides, and amino acids. These simple compounds can support the growth of secondary degraders. Previous studies have demonstrated that pathogens, such as Clostridioides difficile, take advantage of reduced nutrient competition and utilise elevated nutrients in an antibiotic-disturbed gut microbiota.

Nutrient metabolism by the gut microbiota also results in the production of metabolites, some of which may be inhibitory towards gut pathogens. Polysaccharide fermentation results in the generation of metabolites such as short chain fatty acids (SCFAs; e.g. formate, acetate, propionate, butyrate, valerate) and organic acids (e.g. succinate, lactate, ethanol). Protein fermentation results in the production of SCFAs and branched chain fatty acids (BCFAs; e.g. isobutyrate and isovalerate). Gut microbes can use some microbial metabolites as a food source to support their growth (“secondary fermentation”). However, microbial metabolites can also inhibit the growth of some bacteria, including pathogens such as Clostridioides difficile and Salmonella enterica serovar Typhimurium.

In order to design effective therapies to prevent CRE and VRE intestinal colonisation or to decolonise CRE and VRE from the intestine, a better understanding how antibiotics disrupt colonisation resistance to promote CRE and VRE growth is needed.

In view of the problems in the art, there are required new and improved methods for preventing and treating CRE and VRE infections, or intestinal conisations of CRE and/or VRE.

As described in the Examples, the inventors have demonstrated that antibiotics disrupt nutrient metabolism, resulting in an increase in nutrient availability and a decrease in microbial metabolites. They have also shown that these elevated nutrients act as carbon and nitrogen sources to support CRE and VRE growth, where CRE and VRE show an order of preference for specific nutrients. They also show that growth of CRE and VRE was higher on these nutrients in an oxygenated environment, which has been associated with an antibiotic-treated gut microbiota. Accordingly, based on these results, the inventors went on to demonstrate that certain metabolites that were decreased with antibiotics are inhibitory towards CRE or VRE growth. Overall, these data demonstrate that antibiotic-mediated disruption of colonisation resistance involves an increase in nutrient availability and a decrease in the production of inhibitory metabolites that results from the killing of gut commensals.

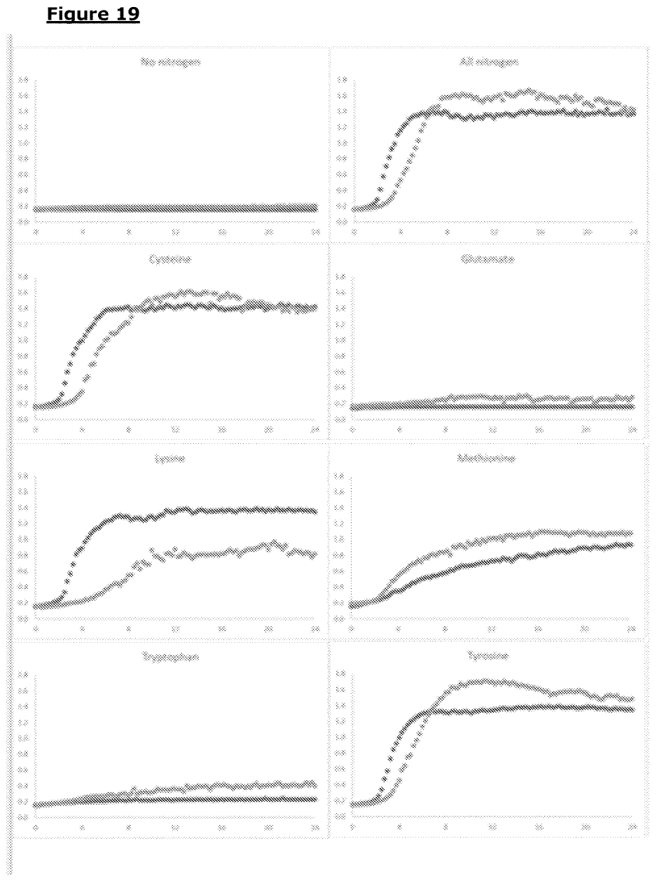

Hence, based on the novel understanding of which nutrients are elevated (see FIGS. 2 and 17), the inventors have designed a novel (i.e., synthetic or artificial) microbial consortium made up of specific members of the gut microbiota (Bifidobacteriaceae; Bacteroidales; and Coriobacteriaceae), which reduce these nutrients in the gut and thereby reduce or suppress growth of CRE and VRE. Furthermore, they have observed that various alcohols (e.g., ethanol), and carboxylate/carboxylic metabolites can also surprisingly prevent CRE/VRE growth in the gut.

Thus, in a first aspect of the invention, there is provided (i) one or more microorganism selected from a group of microorganisms consisting of: Bifidobacteriaceae; Bacteroidales; and Coriobacteriaceae; and/or (ii) one or more compound comprising a short chain alcohol or a C₁-C₁₀ carboxylate or carboxylic acid, wherein the carboxylate or carboxylic acid is optionally substituted with OH and/or NH₂, or a pharmaceutically acceptable salt, solvate, tautomeric form or polymorphic form thereof, for use in treating, preventing or ameliorating an infection or intestinal colonisation of carbapenem-resistant Enterobacteriaceae (CRE) and/or vancomycin-resistant Enterococcus (VRE).

Furthermore, in a second aspect, there is provided a method of treating, preventing or ameliorating an infection or intestinal colonisation of carbapenem-resistant Enterobacteriaceae (CRE) and/or vancomycin-resistant Enterococcus (VRE) in a subject, the method comprising administering to a subject in need of such treatment, a therapeutically active amount of: (i) one or more microorganism selected from a group of microorganisms consisting of: Bifidobacteriaceae; Bacteroidales; and Coriobacteriaceae; and/or (ii) one or more compound comprising a short chain alcohol or a C₁-C₁₀ carboxylate or carboxylic acid, wherein the carboxylate or carboxylic acid is optionally substituted with OH and/or NH₂, or a pharmaceutically acceptable salt, solvate, tautomeric form or polymorphic form thereof.

Surprisingly, as shown in the Examples, the inventors have demonstrated that the one or more compound comprising a short chain alcohol or a C₁-C₁₀ carboxylate or carboxylic acid (herein referred to as an inhibitory metabolite) is able to suppress or cause a reduction in CRE and/or VRE growth. Furthermore, the one or more microorganism selected from a group of microorganisms consisting of: Bifidobacteriaceae; Bacteroidales; and Coriobacteriaceae (herein referred to as a synthetic microbial consortium) can colonise the intestine, and outcompete CRE and/or VRE for nutrients in the gut, and thereby inhibit CRE and/or VRE growth by producing further inhibitory metabolites, resulting in a long-term reduction in CRE and/or VRE growth in the gut.

The one or more microorganism may comprise a microorganism selected from Bifidobacteriaceae. The one or more Bifidobacteriaceae microorganism may be a Bifidobacterium. The one or more Bifidobacteriaceae microorganism is preferably selected from a group consisting of: Bifidobacterium pseudocatenulatum, Bifidobacterium kashiwanohense, Bifidobacterium catenulatum, Bifidobacterium bifidum, Bifidobacterium longum, Bifidobacetrium merycicum, Bifidobacterium angulatum, Bifidobacterium thermacidophilum, Bifidobacterium thermophilum, and Bifidobacterium adolescentis.

Preferably, a plurality of Bifidobacteriaceae microorganisms (more preferably, Bifidobacteria) are used to treat, prevent or ameliorate an infection or intestinal colonisation of carbapenem-resistant Enterobacteriaceae (CRE) and/or vancomycin-resistant Enterococcus (VRE). Preferably, at least 1×10⁶, at least 1×10⁷ or at least 1×10⁸ Bifidobacteriaceae microorganisms or colony forming units (CFU) are used. More preferably, at least 1×10⁹, or at least 1×10¹⁰ Bifidobacteriaceae microorganisms or CFU are used.

The one or more microorganism may comprise a microorganism selected from Bacteroidales. The one or more Bacteroidales microorganism may be a Bacteroides. The one or more Bacteroides microorganism is preferably selected from a group consisting of: Bacteroides pectinophilus, Bacteroides plebius, Bacteroides xylanisolvens, Bacteroides ovatus, Bacteroides intestinalis, Bacteroides eggerthi, Bacteroides coprophilus, Bacteroides dorei, Bacteroides coprocola, Bacteroides vulgatus, Bacteroides uniformis, Bacteroides massiliensis, Bacteroides fragilis, Bacteroides stercoris, Bacteroides thetaiotaomicron, Bacteroides caccae, Barnesiella intestinihominis, Barnesiella propionica, Prevotella copri, Alistipes finegoldii, Alistipes indistinctus, Alistipes inops, Alistipes massiliensis, Alistipes onderdonkii, Alistipes putredinis, Alistipes shahii, Alistipes timonensis, Parabacteroides distasonis, and Parabacteroides merdae.

Preferably, a plurality of Bacteroidales microorganisms (more preferably, Bacteroides) are used to treat, prevent or ameliorate an infection or intestinal colonisation of carbapenem-resistant Enterobacteriaceae (CRE) and/or vancomycin-resistant Enterococcus (VRE). Preferably, at least 1×10⁶, at least 1×10⁷ or at least 1×10⁸ Bacteroidales microorganisms or CFU are used. More preferably, at least 1×10⁹, or at least 1×10¹⁰ Bacteroidales microorganisms or CFU are used.

The one or more microorganism may comprise a microorganism selected from Coriobacteriaceae. The one or more Coriobacteriaceae microorganism may be a Collinsella, preferably selected from a group consisting of: Collinsella aerofaciens, Collinsella tanakaei, Collinsella acetigenes, Collinesella bouchesdurhonensis, Collinsella ihuae, Collinsella ihumii, Collinsella intestinalis, Collinsella massiliensis, Collinsella phocaeensis, Collinsella provencensis, and Collinsella stercoris.

Preferably, a plurality of Coriobacteriaceae microorganisms (more preferably, Collinsella) are used to treat, prevent or ameliorate an infection or intestinal colonisation of carbapenem-resistant Enterobacteriaceae (CRE) and/or vancomycin-resistant Enterococcus (VRE). Preferably, at least 1×10⁶, at least 1×10⁷ or at least 1×10⁸ Coriobacteriaceae microorganisms or CFU are used. More preferably, at least 1×10⁹, or at least 1×10¹⁰ Coriobacteriaceae microorganisms or CFU are used.

Preferably, the one or more microorganism may comprise a microorganism selected from at least two of: Bifidobacteriaceae; Bacteroidales; and Coriobacteriaceae. For example, the one or more microorganism may comprise a microorganism selected from Bifidobacteriaceae and Bacteroidales; or Bifidobacteriaceae and Coriobacteriaceae; or Bacteroidales and Coriobacteriaceae. Preferably, the one or more microorganism may comprise at least two microorganisms selected from: Bifidobacteriaceae; Bacteroidales; and Coriobacteriaceae. For example, the at least two microorganisms may comprise Bifidobacteriaceae and Bacteroidales; or Bifidobacteriaceae and Coriobacteriaceae; or Bacteroidales and Coriobacteriaceae.

However, most preferably the one or more microorganism may comprise a microorganism selected from each of: Bifidobacteriaceae; Bacteroidales; and Coriobacteriaceae. Preferably, the one or more microorganism may comprise all three microorganisms of Bifidobacteriaceae; Bacteroidales; and Coriobacteriaceae. Preferably, a plurality of Bifidobacteriaceae, Bacteroidales, and Coriobacteriaceae microorganisms are used to treat, prevent or ameliorate an infection or intestinal colonisation of carbapenem-resistant Enterobacteriaceae (CRE) and/or vancomycin-resistant Enterococcus (VRE).

The ratio of the amount of Bifidobacteriaceae:Coriobacteriaceae which is used may be at least 1.5:1, preferably at least 5:1, more preferably at least 10:1, even more preferably at least 15:1, and most preferably no more than 18:1.

The ratio of the amount of Bacteroidales:Coriobacteriaceae which is used may be at least 3:1, preferably at least 10:1, more preferably at least 20:1, even more preferably at least 30:1, and most preferably no more than 36:1.

Thus, the ratio of the amount of Bifidobacteriaceae:Bacteroidales:Coriobacteriaceae which is used may be at least about 1.5:3:1, and preferably no more than about 18:36:1.

Preferably, the one or more microorganism is administered orally to a subject in need of treatment or prophyllaxis. Preferably, the one or more microorganism is formulated into a pill or a liquid suspension, which could be contained in a suitable capsule, or the like.

The one or more compound preferably comprises a short chain alcohol. The short chain alcohol may be a C₁-C₇ or a C₁-C₅ alcohol. In some embodiments, the short chain alcohol is a C₁-C₃ alcohol. Accordingly, the short chain alcohol may be methanol, ethanol, propanol or isopropanol.

In a preferred embodiment, the alcohol is ethanol (C₂ alcohol) which is represented herein as Formula I:

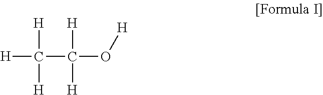

The one or more compound preferably comprises a C₁-C₁₀ carboxylate or carboxylic acid.

The carboxylate or carboxylic acid may be straight or branched chain carboxylate or carboxylic acid.

In some embodiments, the carboxylate or carboxylic acid is substituted with an OH group.

In some embodiments, the carboxylate or carboxylic acid is substituted with an NH₂ group.

The C₁-C₁₀ carboxylate or carboxylic acid may be a short chain fatty acid. The C₁-C₁₀ carboxylate or carboxylic acid may be a long chain fatty acid.

The C₁-C₁₀ carboxylate or carboxylic acid is preferably a C₁-C₇ carboxylate or carboxylic acid, more preferably a C₁-C₆ carboxylate or carboxylic acid, and most preferably a C₁-C₅ carboxylate or carboxylic acid.

Preferably, the carboxylate or carboxylic acid is a C₅ carboxylate or carboxylic acid. In a preferred embodiment, the C₅ carboxylate or carboxylic acid is valerate which is represented herein as Formula II:

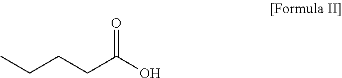

In a preferred embodiment, the C₅ carboxylate or carboxylic acid is isovalerate which is represented herein as Formula III:

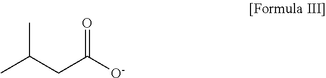

In a preferred embodiment, the C₅ carboxylate or carboxylic acid is 5-aminovalerate (C₅) which is represented herein as Formula IV:

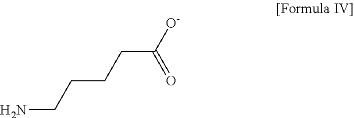

Preferably, the carboxylate or carboxylic acid is a C₄ carboxylate or carboxylic acid. In a preferred embodiment, the C₄ carboxylate or carboxylic acid is butyrate which is represented herein as Formula V:

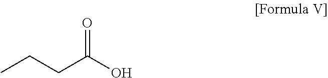

In a preferred embodiment, the C₄ carboxylate or carboxylic acid is isobutyrate which is represented herein as Formula VI:

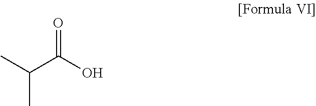

Preferably, the carboxylate or carboxylic acid is a C₃ carboxylate or carboxylic acid. In a preferred embodiment, the C₃ carboxylate or carboxylic acid is propionate which is represented herein as Formula VII:

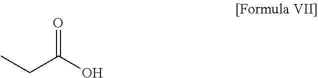

In a preferred embodiment, the C₃ carboxylate or carboxylic acid is lactate which is represented herein as Formula VIII:

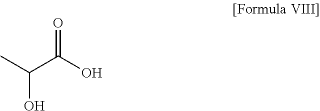

Preferably, the carboxylate or carboxylic acid is a C₂ carboxylate or carboxylic acid. In a preferred embodiment, the C₂ carboxylate or carboxylic acid is acetate which is represented herein as Formula IX:

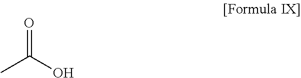

Preferably, the carboxylate or carboxylic acid is a C₁ carboxylate or carboxylic acid. In a preferred embodiment, the C₁ carboxylate or carboxylic acid is formate which is represented herein as Formula X:

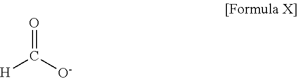

Accordingly, the carboxylate or carboxylic acid may be formate, acetate, propionate, butyrate, isobutyrate, valerate, isovalerate, 5-aminovalerate or lactate.

The skilled person will appreciate that whether or not the one or more compound is a carboxylate or carboxylic acid will greatly depend on the pH. Accordingly, the carboxylate or carboxylic acid may therefore be formate or formic acid, acetate or acetic acid, propionate or propionic acid, butyrate or butyric acid, isobutyrate or isobutyric acid, valerate or valeric acid, isovalerate or isovaleric acid, 5-aminovalerate or aminovaleric acid, or lactate or lactic acid.

Preferably, a plurality of compounds (or “inhibitory metabolites”) comprising a short chain alcohol, or a C₁-C₁₀ carboxylate or carboxylic acid are used to treat, prevent or ameliorate an infection or intestinal colonisation of CRE and/or VRE. Preferably, the plurality of compounds is selected from a group consisting of: (i) ethanol, (ii) formate or formic acid, (iii) acetate or acetic acid, (iv) propionate or propionic acid, (v) butyrate or butyric acid, (vi) isobutyrate or isobutyric acid, (vii) valerate or valeric acid, (viii) isovalerate or isovaleric acid, (ix) 5-aminovalerate or aminovaleric acid, and (x) lactate or lactic acid.

Preferably, at least one, two or three compounds (or inhibitory metabolites) comprising a short chain alcohol or a C₁-C₁₀ carboxylate or carboxylic acid are used to treat, prevent or ameliorate an infection or intestinal colonisation of CRE and/or VRE, more preferably selected from a group consisting of: (i) ethanol, (ii) formate or formic acid, (iii) acetate or acetic acid, (iv) propionate or propionic acid, (v) butyrate or butyric acid, (vi) isobutyrate or isobutyric acid, (vii) valerate or valeric acid, (viii) isovalerate or isovaleric acid, (ix) 5-aminovalerate or aminovaleric acid, and (x) lactate or lactic acid.

Preferably, at least four, five or six compounds (or inhibitory metabolites) comprising a short chain alcohol or a C₁-C₁₀ carboxylate or carboxylic acid are used to treat, prevent or ameliorate an infection or intestinal colonisation of CRE and/or VRE, more preferably selected from a group consisting of: (i) ethanol, (ii) formate or formic acid, (iii) acetate or acetic acid, (iv) propionate or propionic acid, (v) butyrate or butyric acid, (vi) isobutyrate or isobutyric acid, (vii) valerate or valeric acid, (viii) isovalerate or isovaleric acid, (ix) 5-aminovalerate or aminovaleric acid, and (x) lactate or lactic acid.

Preferably, at least seven, eight, or nine compounds (or inhibitory metabolites) comprising a short chain alcohol or a C₁-C₁₀ carboxylate or carboxylic acid are used to treat, prevent or ameliorate an infection or intestinal colonisation of CRE and/or VRE, more preferably selected from a group consisting of: (i) ethanol, (ii) formate or formic acid, (iii) acetate or acetic acid, (iv) propionate or propionic acid, (v) butyrate or butyric acid, (vi) isobutyrate or isobutyric acid, (vii) valerate or valeric acid, (viii) isovalerate or isovaleric acid, (ix) 5-aminovalerate or aminovaleric acid, and (x) lactate or lactic acid.

Preferably, at least 10 compounds (or inhibitory metabolites) comprising a short chain alcohol or a C₁-C₁₀ carboxylate or carboxylic acid are used to treat, prevent or ameliorate an infection or intestinal colonisation of CRE and/or VRE, more preferably selected from a group consisting of: (i) ethanol, (ii) formate or formic acid, (iii) acetate or acetic acid, (iv) propionate or propionic acid, (v) butyrate or butyric acid, (vi) isobutyrate or isobutyric acid, (vii) valerate or valeric acid, (viii) isovalerate or isovaleric acid, (ix) 5-aminovalerate or aminovaleric acid, and (x) lactate or lactic acid.

Preferably, the plurality of compounds comprise ethanol, formate, acetate, propionate, butyrate, isobutyrate, valerate, isovalerate, 5-aminovalerate and lactate.

Preferably, between 0.5 mM and 180 mM of ethanol is administered, more preferably about 10 mM.

Preferably, between 0.05 mM and 0.3 mM of formate is administered, more preferably about 0.10 mM.

Preferably, between 10 mM and 200 mM of acetate is administered, more preferably about 65 mM.

Preferably, between 5 mM and 50 mM of propionate is administered, more preferably about 15 mM.

Preferably, between 3 mM and 50 mM of butyrate is administered, more preferably about 15 mM.

Preferably, between 0.5 mM and 15 mM of valerate is administered, more preferably about 3.5 mM.

Preferably, between 0.5 mM and 6 mM of isobutyrate is administered, more preferably about 2 mM.

Preferably, between 0.5 mM and 6 mM of isovalerate is administered, more preferably about 2 mM.

Preferably, between 0.25 mM and 4 mM of aminovalerate is administered, more preferably about 1 mM.

Preferably, between 0.25 mM and 2 mM of lactate is administered, more preferably about 0.7 mM.

Pharmaceutically acceptable salts include any salt of the compound of Formula (I to (X) provided herein which retains its biological properties and which is not toxic or otherwise undesirable for pharmaceutical use. The pharmaceutically acceptable salt may be derived from a variety of organic and inorganic counter-ions well known in the art.

The pharmaceutically acceptable salt may comprise an acid addition salt formed with organic or inorganic acids such as hydrochloric, hydrobromic, sulfuric, nitric, phosphoric, sulfamic, acetic, trifluoroacetic, trichloroacetic, propionic, hexanoic, cyclopentylpropionic, glycolic, glutaric, pyruvic, lactic, malonic, succinic, sorbic, ascorbic, malic, maleic, fumaric, tartaric, citric, benzoic, 3-(4-hydroxybenzoyl)benzoic, picric, cinnamic, mandelic, phthalic, lauric, methanesulfonic, ethanesulfonic, 1,2-ethane-disulfonic, 2-hydroxyethanesulfonic, benzenesulfonic, 4-chlorobenzenesulfonic, 2-naphthalenesulfonic, 4-toluenesulfonic, camphoric, camphorsulfonic, 4-methylbicyclo[2.2.2]-oct-2-ene-1-carboxylic, glucoheptonic, 3-phenylpropionic, trimethylacetic, tert-butylacetic, lauryl sulfuric, gluconic, benzoic, glutamic, hydroxynaphthoic, salicylic, stearic, cyclohexylsulfamic, quinic, muconic acid and the like acids. Alternatively, the pharmaceutically acceptable salt may comprise a base addition salt formed when an acidic proton present in the parent compound is either replaced by a metal ion, e.g., an alkali metal ion, an alkaline earth ion, an aluminium ion, alkali metal or alkaline earth metal hydroxides, such as sodium, potassium, calcium, magnesium, aluminium, lithium, zinc, and barium hydroxide, or coordinates with an organic base, such as aliphatic, alicyclic, or aromatic organic amines, such as ammonia, methylamine, dimethylamine, diethylamine, picoline, ethanolamine, diethanolamine, triethanolamine, ethylenediamine, lysine, arginine, ornithine, choline, N,N′-dibenzylethylene-diamine, chloroprocaine, diethanolamine, procaine, N-benzylphenethylamine, N-methylglucamine piperazine, tris(hydroxymethyl)-aminomethane, tetramethylammonium hydroxide, and the like.

A pharmaceutically acceptable solvate refers to a compound of Formula (I to X) provided herein, or a salt thereof, that further includes a stoichiometric or non-stoichiometric amount of solvent bound by non-covalent intermolecular forces. Where the solvent is water, the solvate is a hydrate.

The compound of Formula (I) to (X) provided or a pharmaceutically acceptable salt, solvate, tautomeric form or polymorphic form thereof, may be modified to aid delivery to the site of treatment, for example the gut. In one embodiment, the compound of Formula (I) to (X) provided herein may be modified to comprise an inulin ester. In another embodiment, the compound of Formula (I) to (X) may be modified to triacetin, tripropionin, tributyrin, triisobutyrin, triisovalerin, triformin, trivalerin, trihexanoin, triheptanoin, trioctanoin or tripelargonin.

The inventors believe that administering the inhibitory compound (i.e. acetate, propionate, etc.) directly to the patient may be absorbed too quickly by the host (e.g. in the stomach) before the compound has had a chance to reach the large intestine where it needs to imparts its inhibitory effects. Therefore, in another embodiment, the compound may be attached to, or conjugated with, a carrier molecule. The carrier molecule improves the delivery of the compound to the large intestine (i.e. the gut) where it imparts its effects.

The carrier molecule may comprise glycerol or a glycerol backbone. For example, the glycerol backbone may be represented herein as Formula XI:

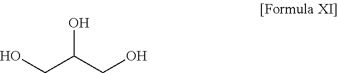

The conjugation may be achieved by a process described in CN105218366, the contents of which are incorporated herein by reference. Thus, the conjugated compound (i.e. comprising the carrier molecule) may comprise glycerol triacetate, glycerol tri propionate, glycerol tri formate, glycerol tributyrate, glycerol trivalerate, glycerol triisovalerate, glycerol triisobutyrate, glycerol trilactate, glycerol tri-5-aminovalerate, and/or glycerol triethanol.

Accordingly, administering the compound conjugate to glycerol (e.g. glycerol triacetate, glycerol tripropionate, etc.) means that they are more likely to reach the large intestine, where the short chain fatty acids etc. are hydrolysed by lipases and released into the large intestine where then can inhibit the CRE/VRE.

In one embodiment, either (i) one or more microorganism selected from a group of microorganisms consisting of: Bifidobacteriaceae; Bacteroidales; and Coriobacteriaceae; or (ii) one or more compound comprising a short chain alcohol or a C₁-C₁₀ carboxylate or carboxylic acid, wherein the carboxylate or carboxylic acid is optionally substituted with OH and/or NH₂, or a pharmaceutically acceptable salt, solvate, tautomeric form or polymorphic form thereof, is used to treat, prevent or ameliorate an infection or intestinal colonisation of CRE and/or VRE.

Preferably, the one or more microorganism, or the one or more compound, is used to treat, prevent or ameliorate colonisation of CRE and/or VRE in the gastrointestinal (GI) tract, most preferably before they cause an invasive infection, for example in the bloodstream.

In a preferred embodiment, however, (i) one or more microorganism selected from a group of microorganisms consisting of: Bifidobacteriaceae; Bacteroidales; and Coriobacteriaceae; and (ii) one or more compound comprising a short chain alcohol or a C₁-C₁₀ carboxylate or carboxylic acid, wherein the carboxylate or carboxylic acid is optionally substituted with OH and/or NH₂, or a pharmaceutically acceptable salt, solvate, tautomeric form or polymorphic form thereof, are both used to treat, prevent or ameliorate an infection or intestinal colonisation of CRE and/or VRE. The (i) one or more microorganism, and the (ii) one or more compound comprising a short chain alcohol or a C₁-C₁₀ carboxylate or carboxylic acid, may be administered simultaneously. Alternatively, the (i) one or more microorganism, and the (ii) one or more compound comprising a short chain alcohol or a C₁-C₁₀ carboxylate or carboxylic acid, may be administered sequentially (i.e. separately).

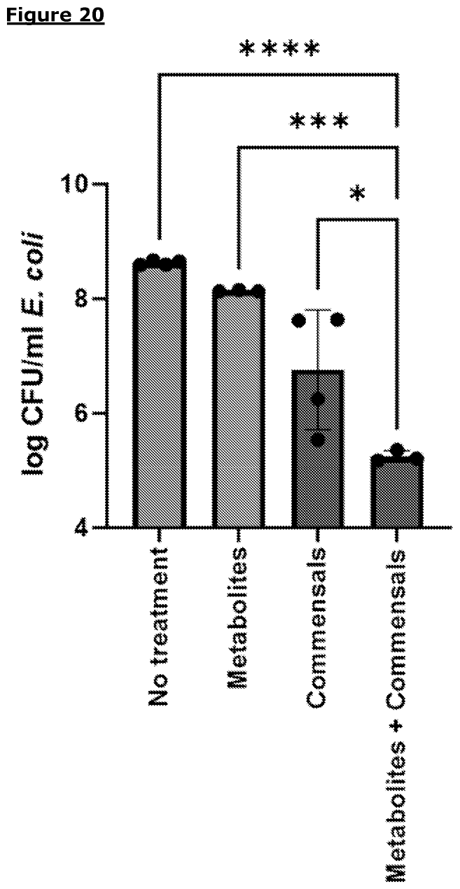

As shown in FIG. 20, the inventors have clearly demonstrated that a combination of commensal gut microorganisms and inhibitory metabolites suppresses the growth of carbapenem-resistant E. coli, and that the combination of commensal gut microorganisms and inhibitory metabolites was more suppressive to the growth of CRE than either mixture alone. Advantageously, combining both the inhibitory metabolites and the microbial consortium to treat an infection or intestinal colonisation of CRE and/or VRE results in an improved inhibition of the CRE and/or VRE. In use, preferably the one or more compound comprising a short chain alcohol or a C₁-C₁₀ carboxylate or carboxylic acid is administered to the patient first, and thereby suppresses and causes a reduction in CRE and/or VRE growth, and then the one or more microorganism selected from a group of microorganisms consisting of: Bifidobacteriaceae; Bacteroidales; and Coriobacteriaceae is administered to the patient, such that the microorganism colonises the intestine, and outcompetes CRE and/or VRE for nutrients in the gut, thereby inhibiting the CRE and/or VRE growth by producing further inhibitory metabolites, resulting in a long-term reduction in CRE and/or VRE growth in the gut.

Preferably, an infection or intestinal colonisation of CRE or VRE is treated, prevented or ameliorated. However, more preferably an infection or intestinal colonisation of CRE and VRE is treated, prevented or ameliorated.

The carbapenem-resistant Enterobacteriaceae (CRE) may be carbapenem-resistant Enterobacteriaceae. The carbapenem-resistant Enterobacteriaceae (CRE) may be selected from a group consisting of carbapenem-resistant Escherichia, Klebsiella, Enterobacter, Citrobacter, Proteus, Serratia, and Salmonella.

The carbapenem-resistant Enterobacteriaceae (CRE) may be selected from a group consisting of carbapenem-resistant Escherichia coli, Klebsiella pneumoniae, Klebsiella oxytoca, Klebsiella aerogenes, Enterobacter cloacae, Enterobacter cloacae complex (which encompasses Enterobacter asburiae, Enterobacter kobei, Enterobacter ludwigii, Enterobacter hormaechei subsp. oharae, subsp. hormaechei, and subsp. steigerwaltii, Enterobacter nimipressuralis, E. cloacae subsp. cloacae and subsp. dissolvens), Enterobacter gergoviae, Citrobacter freundii, Proteus mirabilis, Salmonella enterica, and Serratia marcescens.

Preferably, the carbapenem-resistant Enterobacteriaceae (CRE) may be carbapenem-resistant Escherichia coli, Klebsiella pneumoniae, and/or Enterococcus cloacae. The CRE may be carbapenem-resistant E. coli, K. pneumoniae, and E. cloacae.

The vancomycin-resistant Enterococcus (VRE) may be vancomycin-resistant Enterococcaceae. The vancomycin-resistant Enterococcus (VRE) may be vancomycin-resistant Enterococcus.

The vancomycin-resistant Enterococcus (VRE) may be selected from a group consisting of vancomycin-resistant Enterococcus faecalis, Enterococcus faecium, Enterococcus gallinarum, Enterococcus casseliflavus, Enterococcus durans, Enterococcus avium, and Enterococcus raffinosis.

The vancomycin-resistant Enterococcus (VRE) may be vancomycin-resistant Enterococcus faecalis and/or Enterococcus faecium. The VRE may be vancomycin-resistant E. faecalis and E. faecium.

Thus, in a preferred embodiment, the invention provides (i) a plurality of microorganisms comprising Bifidobacteriaceae; Bacteroidales; and Coriobacteriaceae; and (ii) a plurality of compounds selected from a group consisting of: ethanol, formate, acetate, propionate, butyrate, isobutyrate, valerate, isovalerate, 5-aminovalerate and lactate, or a pharmaceutically acceptable salt, solvate, tautomeric form or polymorphic form thereof, for use in treating, preventing or ameliorating an infection or intestinal colonisation of carbapenem-resistant Enterobacteriaceae (CRE) and/or vancomycin-resistant Enterococcus (VRE).

In a more preferred embodiment, the invention provides a novel microbiome-based therapeutic which comprises:
(a) a synthetic microbial consortium selected from Bifidobacteriaceae (e.g. Bifidobacterium), Bacteroidales (e.g. Bacteroides) and Coriobacteriaceae (e.g. Collinsella); and
(b) one or more inhibitory metabolites selected from formate, acetate, propionate, butyrate, isobutyrate, valerate, isovalerate, 5-aminovalerate, lactate and ethanol.

The one or more microorganism selected from a group of microorganisms consisting of: Bifidobacteriaceae; Bacteroidales; and Coriobacteriaceae is referred to herein as the microbial consortium. The one or more compound comprising a short chain alcohol or a C₁-C₁₀ carboxylate or carboxylic acid is referred to herein as the inhibitory compound or metabolite. The microbial consortium and the inhibitory compound may be used separately or in combination for treating a CRE or VRE infection.

It will be appreciated that the microbial consortium and the inhibitory compound according to the invention may be used in a monotherapy, i.e., the sole use of the microbial consortium and/or the inhibitory compound, for treating, ameliorating or preventing a CRE or VRE infection. Alternatively, the microbial consortium and the inhibitory compound according to the invention may be used as an adjunct to, or in combination with, known therapies for treating, ameliorating, or preventing a CRE or VRE infection. For example, the agent may be used in combination with known agents for treating a CRE or VRE infections. Antibiotics used for a CRE or VRE infections include carbapenems, polymyxins, aminoglycosides, tigecycline, fosfomycin, and/or beta-lactam/beta-lactamase inhibitors.

The microbial consortium and inhibitory compound according to the invention may be combined in compositions having a number of different forms depending, in particular, on the manner in which the composition is to be used. Thus, for example, the composition may be in the form of a pill, powder, tablet, capsule, liquid or any other suitable form that may be administered to a person or animal in need of treatment. It will be appreciated that the vehicle of medicaments according to the invention should be one which is well-tolerated by the subject to whom it is given.

The CRE and/or VRE treatment compositions and formulations of the invention may be used in a number of ways. For instance, oral administration may be required, in which case the agents may be contained within a composition that may, for example, be ingested orally in the form of a tablet, capsule or liquid. Preferably, therefore, the microbial consortium and inhibitory compound according to the invention is administered orally to the patient. The composition may be slow release, such as slow release tablet.

It will be appreciated that the amount of the CRE and/or VRE treatment compositions and formulations that is required is determined by its biological activity and bioavailability, which in turn depends on the mode of administration, the physiochemical properties of the CRE and/or VRE treatment compositions and formulations, and whether they are being used as a monotherapy or in a combined therapy. The frequency of administration will also be influenced by the half-life of the CRE and/or VRE treatment compositions and formulations within the subject being treated. Optimal dosages to be administered may be determined by those skilled in the art, and will vary with the particular CRE and/or VRE treatment compositions and formulations in use, the strength of the pharmaceutical composition, the mode of administration, and the advancement of the bacterial infection. Additional factors depending on the particular subject being treated will result in a need to adjust dosages, including subject age, weight, gender, diet, and time of administration.

Generally, a daily dose of between 0.001 μg/kg of body weight and 10 mg/kg of body weight, or between 0.01 μg/kg of body weight and 1 mg/kg of body weight, of CRE and/or VRE treatment composition or formulation according to the invention may be used for treating, ameliorating, or preventing bacterial infection, depending upon which CRE and/or VRE treatment composition or formulation is used. Preferably, inhibitory compound or a pharmaceutically acceptable salt or solvate thereof is applied at a rate of at least 1 mM in the intestinal content, more preferably at least 10 mM in the intestinal content, most preferably at least 30 mM in the intestinal content.

Preferably, at least 1×10⁶, at least 1×10⁷, at least 1×10⁸, at least 1×10⁹, or at least 1×10¹⁰ microorganisms or CFU are administered.

Preferably, the following inhibitory compounds or a pharmaceutically acceptable salt or solvate thereof is administered (e.g. in the intestinal content) at a rate of: (i) between 0.5 mM and 180 mM of ethanol, more preferably about 10 mM of ethanol; (ii) between 0.05 mM and 0.3 mM of formate, more preferably about 0.10 mM formate; (iii) between 10 mM and 200 mM of acetate, more preferably about 65 mM acetate; (iv) between 5 mM and 50 mM of propionate, more preferably about 15 mM propionate; (v) between 3 mM and 50 mM of butyrate, more preferably about 15 mM butyrate; (vi) between 0.5 mM and 15 mM of valerate, more preferably about 3.5 mM valerate; (vii) between 0.5 mM and 6 mM of isobutyrate, more preferably about 2 mM isobutyrate; (viii) between 0.5 mM and 6 mM of isovalerate, more preferably about 2 mM isovalerate; (ix) between 0.25 mM and 4 mM of aminovalerate, more preferably about 1 mM aminovalerate; and (x) between 0.25 mM and 2 mM of lactate, more preferably about 0.7 mM lactate.

The CRE and/or VRE treatment composition or formulation may be administered before, during or after onset of the CRE and/or VRE bacterial infection. Daily doses may be given as a single administration (e.g., a single daily pill or capsule or tablet). Alternatively, the CRE and/or VRE treatment composition or formulation may require administration twice or more times during a day. As an example, the CRE and/or VRE treatment composition or formulation may be administered as one or two (or more depending upon the severity of the bacterial infection being treated) daily doses of between 0.07 g and 700 mg (i.e., assuming a body weight of 70 kg). A patient receiving treatment may take a first dose upon waking and then a second dose in the evening (if on a two dose regime) or at 3- or 4-hourly intervals thereafter. Alternatively, a slow release device may be used to provide optimal doses of CRE and/or VRE treatment composition or formulation according to the invention to a patient without the need to administer repeated doses.

It will be appreciated that patients tend to get infected with CRE and/or VRE when they are in hospital and taking antibiotics. Accordingly, it is preferred that the CRE and/or VRE treatment compositions or formulations of the invention are administered prior to hospital entry (e.g., as prescribed over the counter), and then during the stay at hospital, and for a few days or weeks post-discharge. The CRE and/or VRE treatment composition or formulation may therefore be used to prevent relapse of the bacterial infection.

Known procedures, such as those conventionally employed by the pharmaceutical industry (e.g., in vivo experimentation, clinical trials, etc.), may be used to form specific formulations of the CRE and/or VRE treatment composition according to the invention and precise therapeutic regimes (such as daily doses of the antibiotic composition or formulation and the frequency of administration).

According to a third aspect of the invention, there is provided a carbapenem-resistant Enterobacteriaceae (CRE) and/or vancomycin-resistant Enterococcus (VRE) treatment pharmaceutical composition comprising (i) one or more microorganism selected from a group of microorganisms consisting of: Bifidobacteriaceae; Bacteroidales; and Coriobacteriaceae; and/or (ii) one or more compound comprising a short chain alcohol or a C₁-C₁₀ carboxylate or carboxylic acid, wherein the carboxylate or carboxylic acid is optionally substituted with OH and/or NH₂, or a pharmaceutically acceptable salt, solvate, tautomeric form or polymorphic form thereof, and a pharmaceutically acceptable vehicle.

According to a fourth aspect, there is provided a method of preparing the pharmaceutical composition according to the third aspect, the method comprising contacting (i) one or more microorganism selected from a group of microorganisms consisting of: Bifidobacteriaceae; Bacteroidales; and Coriobacteriaceae; and/or (ii) one or more compound comprising a short chain alcohol or a C₁-C₁₀ carboxylate or carboxylic acid, wherein the carboxylate or carboxylic acid is optionally substituted with OH and/or NH₂, or a pharmaceutically acceptable salt, solvate, tautomeric form or polymorphic form thereof with a pharmaceutically acceptable vehicle.

A “subject” may be a vertebrate, mammal, or domestic animal. Hence, compositions and medicaments according to the invention may be used to treat any mammal, for example livestock (e.g. a horse), pets, or may be used in other veterinary applications. Most preferably, however, the subject is a human being.

A “therapeutically effective amount” of the polypeptide or the pharmaceutical composition is any amount which, when administered to a subject, is the amount of the aforementioned that is needed to treat or prevent CRE and/or VRE infection.

For example, the therapeutically effective amount may be from about 0.001 ng to about 1 mg, and preferably from about 0.01 ng to about 100 ng. It is preferred that the amount of agent is an amount from about 0.1 ng to about 10 ng, and most preferably from about 0.5 ng to about 5 ng.

A “pharmaceutically acceptable vehicle” as referred to herein, is any known compound or combination of known compounds that are known to those skilled in the art to be useful in formulating pharmaceutical compositions.

In one embodiment, the pharmaceutically acceptable vehicle may be a solid, and the composition may be in the form of a powder or tablet. A solid pharmaceutically acceptable vehicle may include one or more substances which may also act as flavouring agents, lubricants, solubilisers, suspending agents, dyes, fillers, glidants, compression aids, inert binders, sweeteners, preservatives, dyes, coatings, or tablet-disintegrating agents. The vehicle may also be an encapsulating material. In powders, the vehicle is a finely divided solid that is in admixture with the finely divided active agents according to the invention. In tablets, the active agent may be mixed with a vehicle having the necessary compression properties in suitable proportions and compacted in the shape and size desired. The powders and tablets preferably contain up to 99% of the active agents. Suitable solid vehicles include, for example, calcium phosphate, magnesium stearate, talc, sugars, lactose, dextrin, starch, gelatin, cellulose, polyvinylpyrrolidine, low melting waxes and ion exchange resins.

However, the pharmaceutical vehicle may be a liquid, and the pharmaceutical composition is in the form of a solution. Liquid vehicles are used in preparing solutions, suspensions, emulsions, syrups, elixirs and pressurized compositions. The active agent according to the invention may be dissolved or suspended in a pharmaceutically acceptable liquid vehicle such as water, an organic solvent, a mixture of both or pharmaceutically acceptable oils or fats. The liquid vehicle can contain other suitable pharmaceutical additives such as solubilisers, emulsifiers, buffers, preservatives, sweeteners, flavouring agents, suspending agents, thickening agents, colours, viscosity regulators, stabilizers or osmo-regulators. Suitable examples of liquid vehicles for oral administration include water (partially containing additives as above, e.g., cellulose derivatives, preferably sodium carboxymethyl cellulose solution), alcohols (including monohydric alcohols and polyhydric alcohols, e.g., glycols) and their derivatives, and oils (e.g., fractionated coconut oil and arachis oil).

Liquid pharmaceutical compositions, which are sterile solutions or suspensions, can be utilized by, for example, as a liquid drink or oral suspension. The agent may be prepared as a sterile solid composition that may be dissolved or suspended at the time of administration using sterile water, saline, or other appropriate sterile medium.

The agents and compositions of the invention may be administered orally in the form of a sterile solution or suspension containing other solutes or suspending agents (for example, enough saline or glucose to make the solution isotonic), bile salts, acacia, gelatin, sorbitan monoleate, polysorbate 80 (oleate esters of sorbitol and its anhydrides copolymerized with ethylene oxide) and the like. The agents used according to the invention can also be administered orally either in liquid or solid composition form. Compositions suitable for oral administration include solid forms, such as pills, capsules, granules, tablets, and powders, and liquid forms, such as solutions, syrups, elixirs, and suspensions.

In one embodiment, the pharmaceutically acceptable vehicle may be in the form of a powder, tablet, capsule, liquid, or any other suitable form that may be administered to a person or animal in need of treatment. It will be appreciated that the vehicle of medicaments according to the invention should be one which is well-tolerated by the subject to whom it is given.

In a preferred embodiment, however, the microbial consortium and inhibitory compounds of the invention may be administered directly to the gut.

All of the features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made, by way of example, to the accompanying Figures, in which:—

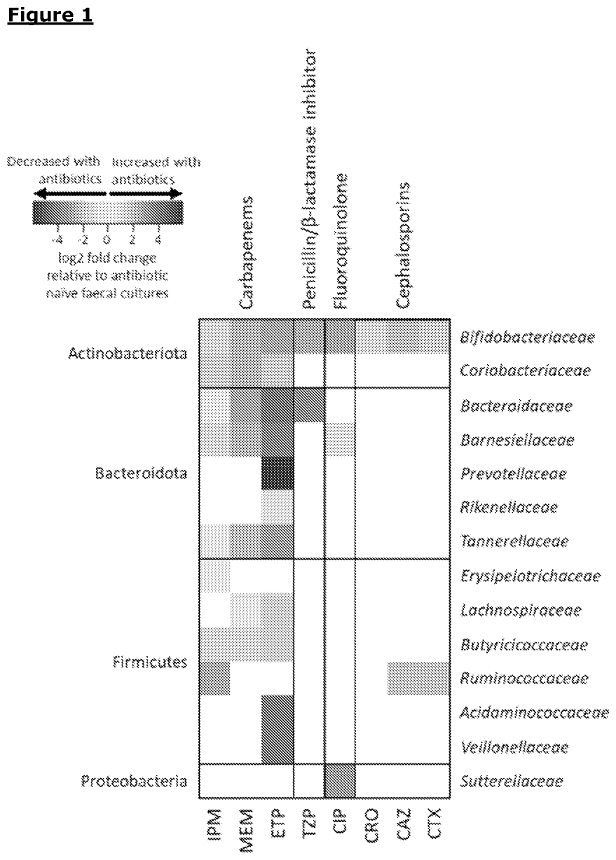

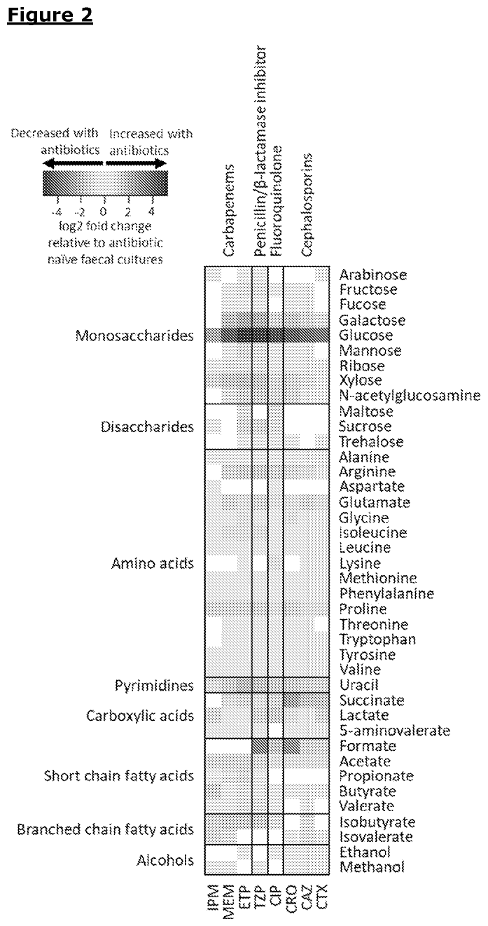

FIG. 1 shows that antibiotic treatment of faecal cultures caused a significant decrease in the abundance of several bacterial families. Log 2 fold change in bacterial families significantly decreased with antibiotics (blue) or significantly increased with antibiotics (red), relative to antibiotic-naïve faecal cultures. Bacterial families that were not significantly changed with antibiotics were not plotted (white). n=11 healthy faecal donors. Wilcoxon signed rank test of log-transformed abundances with Benjamini Hochberg FDR, p<0.05.

FIG. 2 shows that antibiotic treatment of faecal cultures resulted in a significant increase in the concentration of nutrients and a significant decrease in the concentration of microbial metabolites. Log 2 fold change in nutrients and metabolites significantly increased with antibiotics (red) and nutrients and metabolites significantly decreased with antibiotics (blue), relative to antibiotic-naïve faecal cultures. Nutrients and metabolites that were not significantly changed with antibiotics were not plotted (white). n=11 healthy faecal donors. Wilcoxon signed rank test with Benjamini Hochberg FDR, p<0.05.

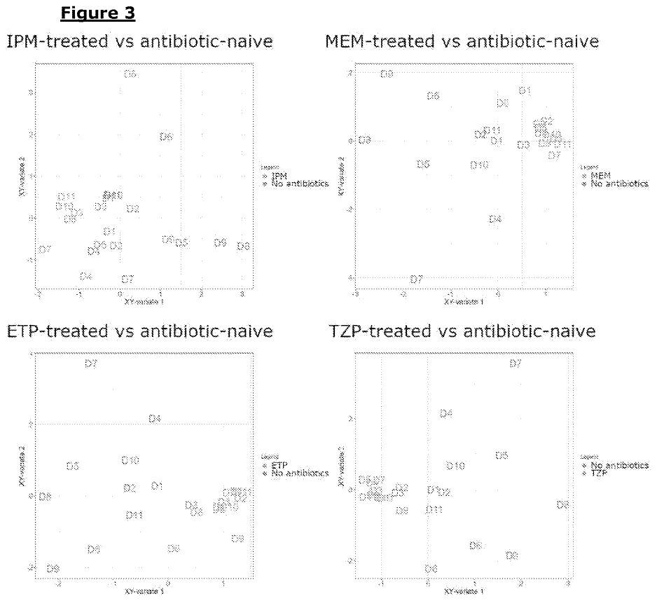

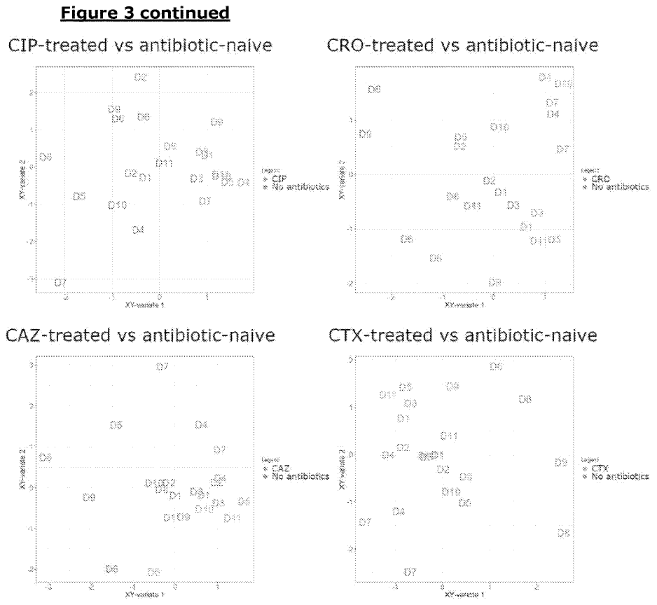

FIG. 3 shows that antibiotic treatment resulted in a separation of antibiotic-treated and untreated faecal culture samples in rCCA representation unit plots. rCCA models correlating 16S rRNA gene sequencing data (family level) and 1H-NMR spectroscopy data. Representation of units (or samples) for the first 2 canonical variates showing the correlations between variables antibiotic-treated (orange) and untreated (blue). D1-D11=Donor number 1-11.

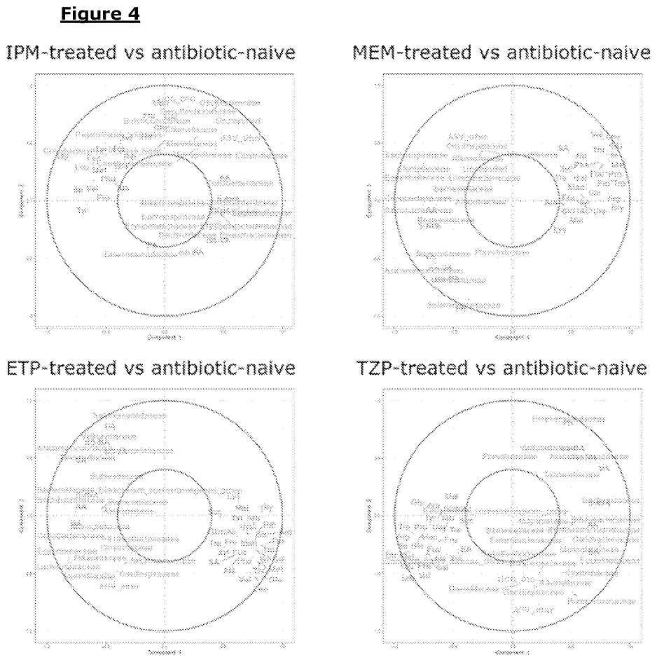

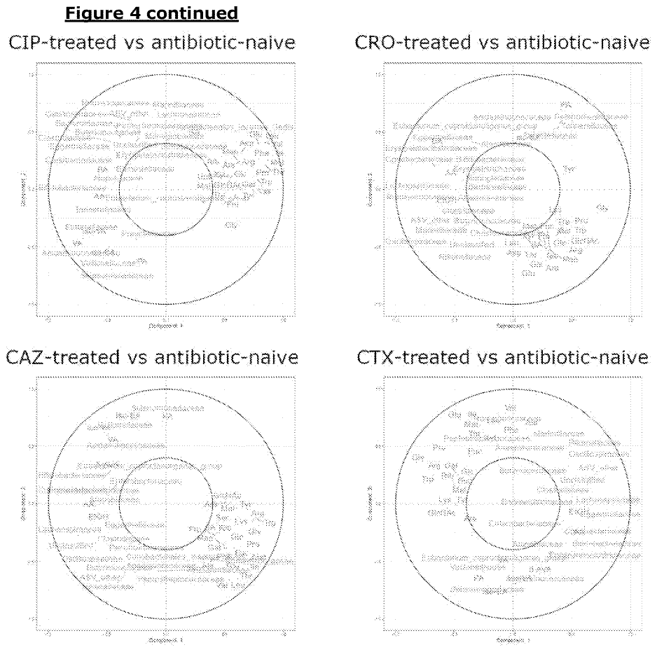

FIG. 4 shows bacterial families that were decreased with antibiotic treatment were positively correlated with microbial metabolites and negatively correlated with nutrients in faecal culture samples. rCCA model correlating 16S rRNA gene sequencing data (family level) and ¹H-NMR spectroscopy data. Correlation circle plot showing correlations between variables from antibiotic-treated and untreated samples. Nutrients and metabolites are shown in blue and bacterial families are shown in orange. The following abbreviations are used for nutrients and metabolites: arabinose, Ara; fructose, Fru; fucose, Fuc; galactose, Gal; glucose, Glc; mannose, Man; ribose, Rib; xylose, Xyl; N-acetylglucosamine, GlcNAc; maltose, Mal; sucrose, Suc; trehalose, Tre; alanine, Ala; arginine, Arg; aspartate, Asp; glutamate, Glu; glycine, Gly; isoleucine, Ile; leucine, Leu; lysine, Lys; methionine, Met; phenylalanine, Phe; proline, Pro; threonine, Thr; tryptophan, Trp; tyrosine, Tyr; valine, Val; uracil, Ura; succinate, SA; lactate, LA; 5-aminovalerate, 5-AVA; formate, FA; acetate, AA; propionate, PA; butyrate, BA; valerate, VA; isobutyrate, iso-BA; isovalerate, iso-VA; ethanol, EtOH.

FIG. 5 shows that CRE growth is supported by a mixture of nutrients that were elevated after antibiotic treatment of faecal microbiota. AMiGA-predicted growth curves for E. coli, K. pneumoniae, and E. cloacae grown on minimal medium supplemented with a mixture of nutrients that were elevated in faecal cultures following antibiotic treatment (or water as the no nutrient control) under aerobic or anaerobic conditions. Growth of each isolate was measured with 6 wells in 2 independent experiments. The predicted mean of growth is shown with bold lines and the predicted 95% credible intervals are shown with the shaded bands.

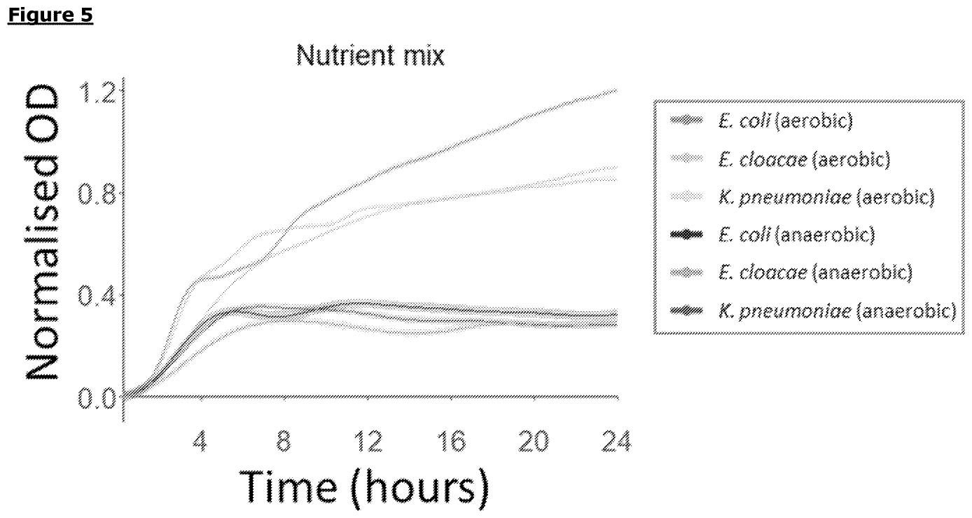

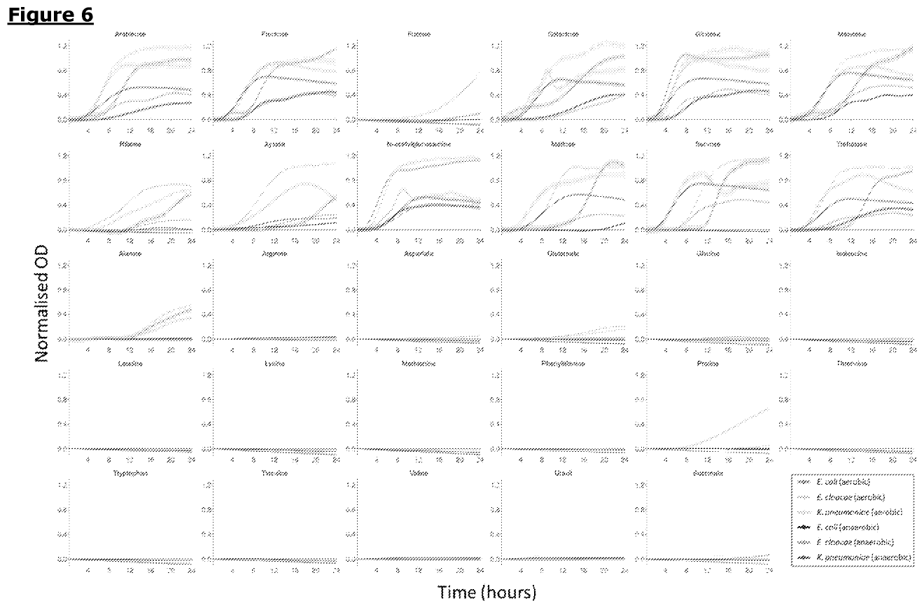

FIG. 6 shows that CRE growth is supported by individual carbon sources found to be elevated after antibiotic treatment. AMiGA-predicted growth curves for E. coli, K. pneumoniae, and E. cloacae grown on M9 minimal medium supplemented with a single carbon source (or water as the no carbon control) under aerobic or anaerobic conditions. Growth of each isolate was measured with 6 wells in 2-3 independent experiments. The predicted mean of growth is shown with bold lines and the predicted 95% credible intervals are shown with the shaded bands.

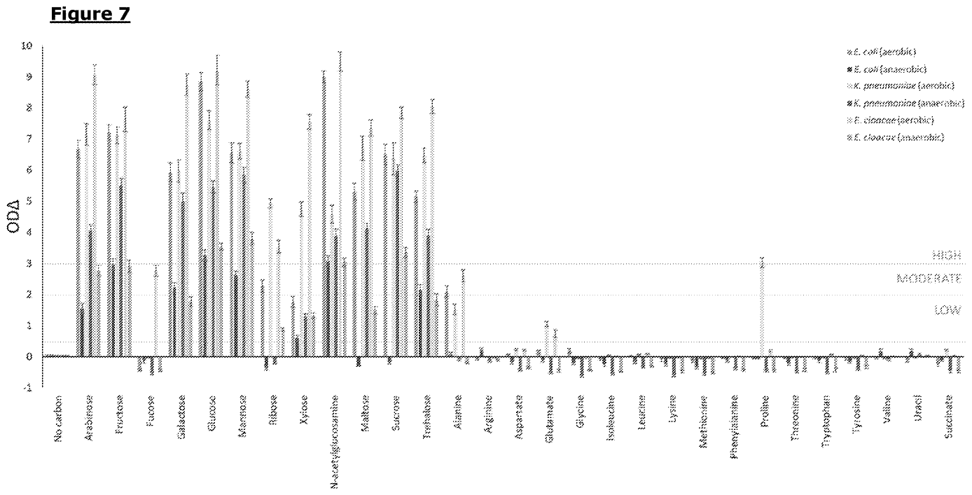

FIG. 7 shows that individual carbon sources support the growth of CRE at high, moderate, or low levels. Functional differences between growth on carbon source versus no carbon control for E. coli, K. pneumoniae, and E. cloacae grown under aerobic or anaerobic conditions are summarized with the sum of functional differences (ODA) quantifying the magnitude of differences between two growth curves. ODΔ>3 indicate high growth, ODΔ between 2-3 indicated moderate growth, ODΔ between 0.5-2 indicate low growth, ODΔ<0.5 indicate negligible or no growth. Error bars indicate 95% confidence intervals.

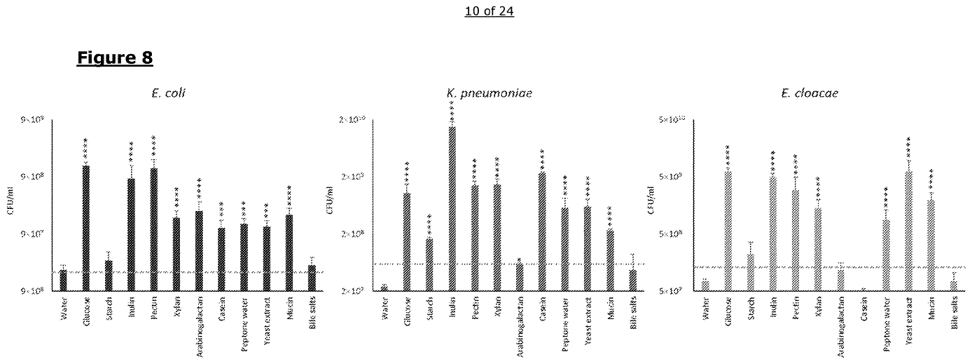

FIG. 8 shows that CRE can utilise some complex substrates as carbon sources to support their growth. Plate counts for E. coli, K. pneumoniae, and E. cloacae grown on M9 minimal medium supplemented with a single complex substrate (water as the no carbon control, or glucose as the positive control) under anaerobic conditions for 24 hours. The dashed grey line shows the starting concentration of the CRE isolate at 0 hours. One-way ANOVA followed by Dunn's multiple comparison test (each substrate was compared to the water control). *=P≤0.05, **=P≤0.01, ***=P≤0.001, ****=P≤0.0001, n=3 wells per isolate.

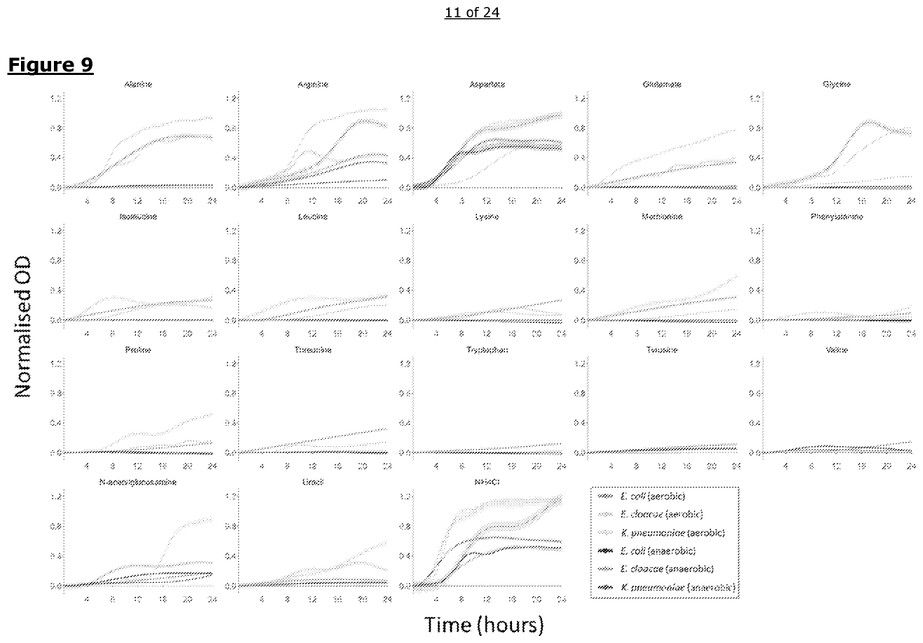

FIG. 9 shows that CRE growth is supported by individual nitrogen sources found to be elevated after antibiotic treatment. AMiGA-predicted growth curves for E. coli, K. pneumoniae, and E. cloacae grown on M9 minimal medium supplemented with a single nitrogen source (or water as the no nitrogen control) under aerobic or anaerobic conditions. Growth of each isolate was measured with 6 wells in 2-3 independent experiments. The predicted mean of growth is shown with bold lines and the predicted 95% credible intervals are shown with the shaded bands. NH4Cl was used as a positive control for growth as a sole nitrogen source.

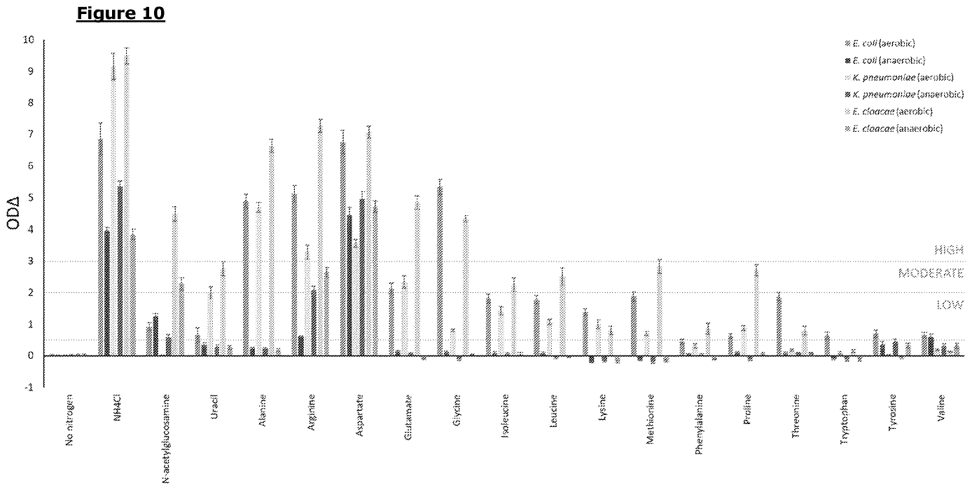

FIG. 10 shows that individual nitrogen sources support the growth of CRE at high, moderate, or low levels. Functional differences between growth on nitrogen source versus no nitrogen control for E. coli, K. pneumoniae, and E. cloacae grown under aerobic or anaerobic conditions are summarized with the sum of functional differences (ODΔ) quantifying the magnitude of differences between two growth curves. ODΔ>3 indicate high growth, ODΔ between 2-3 indicated moderate growth, ODΔ between 0.5-2 indicate low growth, ODΔ<0.5 indicate negligible or no growth. Error bars indicate 95% confidence intervals.

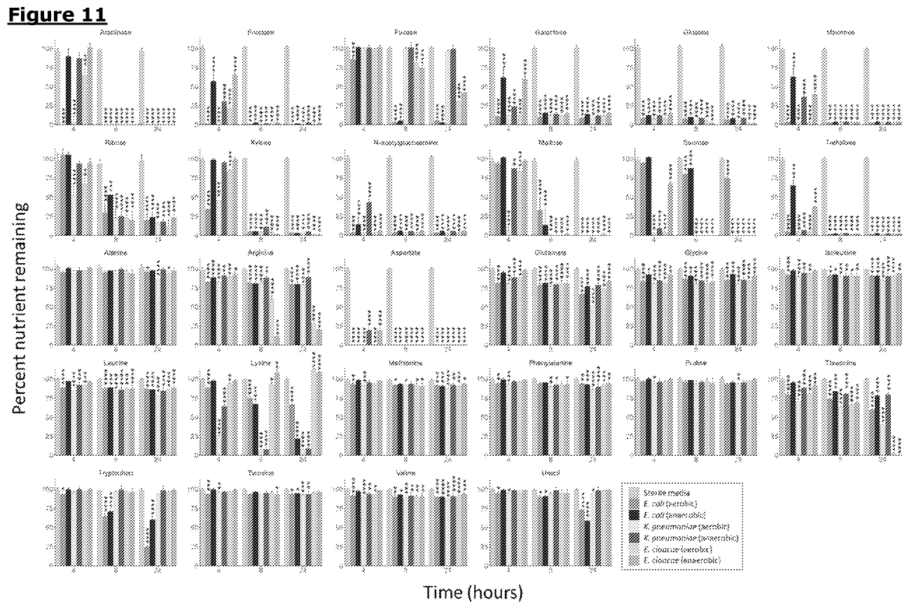

FIG. 11 shows that CRE nutrient utilisation varies between different CRE isolates and for the same CRE isolate grown under aerobic and anaerobic conditions. Percent nutrient remaining for E. coli, K. pneumoniae, or E. cloacae following incubation in M9 minimal medium containing a mixture of 0.015% of each nutrient under anaerobic or aerobic conditions. Nutrient concentration was measured by 1H-NMR spectrometry. The starting nutrient concentration was measured by the integration of a representative peak in the NMR spectrum and the starting nutrient concentration was set to 100% at 0 hrs. Two-way mixed ANOVA followed by pairwise comparisons with Bonferroni correction. *=P 0.05, **=P 0.01, ***=P<0.001, ****=P<0.0001, n=3 wells per isolate.

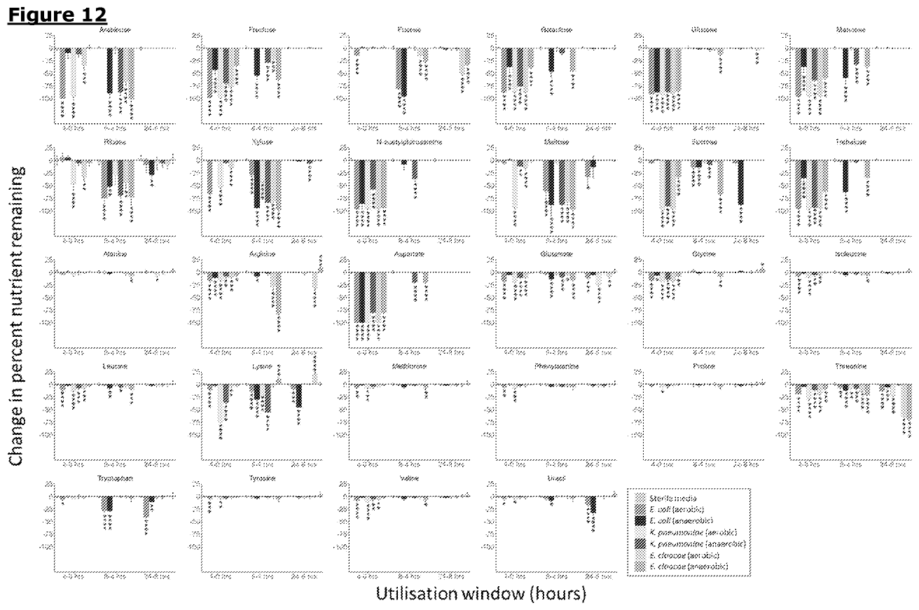

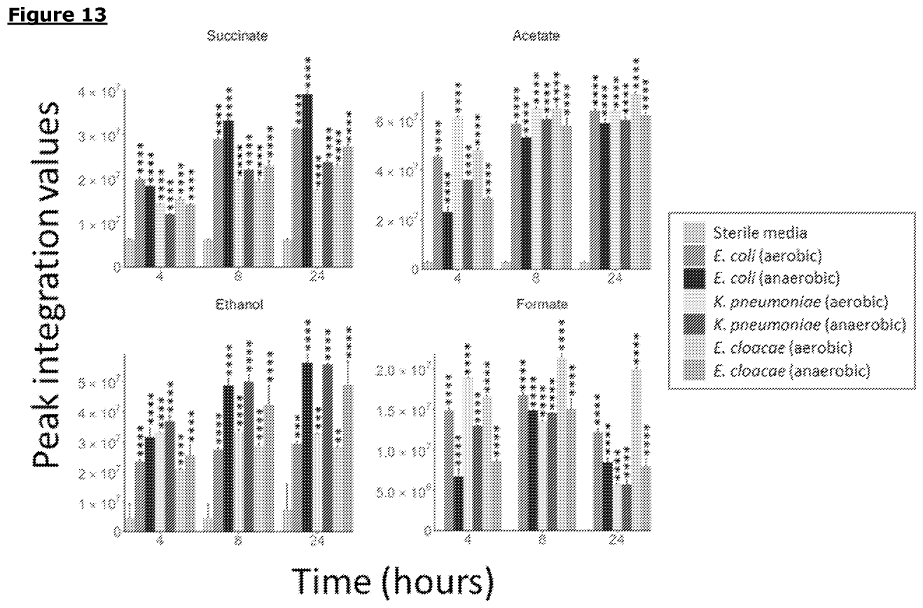

FIG. 12 shows that the order of nutrient utilisation varies by CRE isolate and under anaerobic and aerobic conditions. Percent change in nutrients between subsequent time points by E. coli, K. pneumoniae, or E. cloacae grown under anaerobic or aerobic conditions in M9 minimal medium containing a mixture of 0.015% of each nutrient. Nutrient concentration was measured by 1H-NMR spectrometry. The starting nutrient concentration was measured by the integration of a representative peak in the NMR spectrum and the starting nutrient concentration was set to 100% at 0 hrs. Change in the percent of the nutrient remaining was calculated by subtracting the percent nutrient remaining at the time point from the percent nutrient remaining at the previous time point. Two-way mixed ANOVA followed by pairwise comparisons with Bonferroni correction. *=P≤0.05, **=P≤0.01, ***=P≤0.001, ****=P≤0.0001, n=3 wells per isolate.

FIG. 13 shows that CRE isolates produce metabolites following incubation with a mixture of nutrients. Metabolite production by E. coli, K. pneumoniae, or E. cloacae grown under anaerobic or aerobic conditions in a minimal medium containing a mixture of 0.015% of each nutrient. Metabolite production was measured by 1H-NMR spectrometry. The metabolite concentration was measured by the integration of a representative peak in the NMR spectrum after incubation for 4 hrs, 8 hrs, and 24 hours. Two-way mixed ANOVA followed by pairwise comparisons with Bonferroni correction. *=P≤0.05, **=P≤0.01, ***=P≤0.001, ****=P≤0.0001, n=3 wells per isolate.

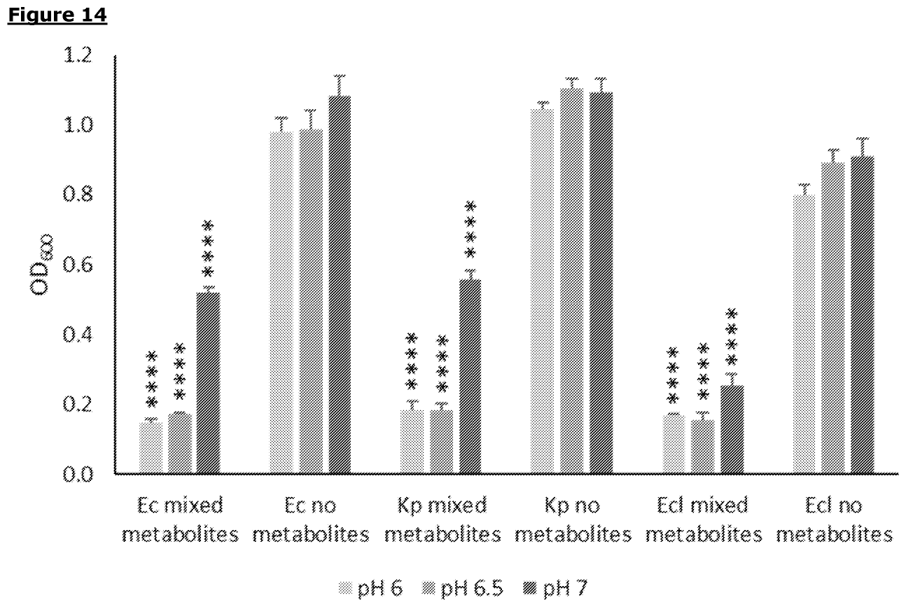

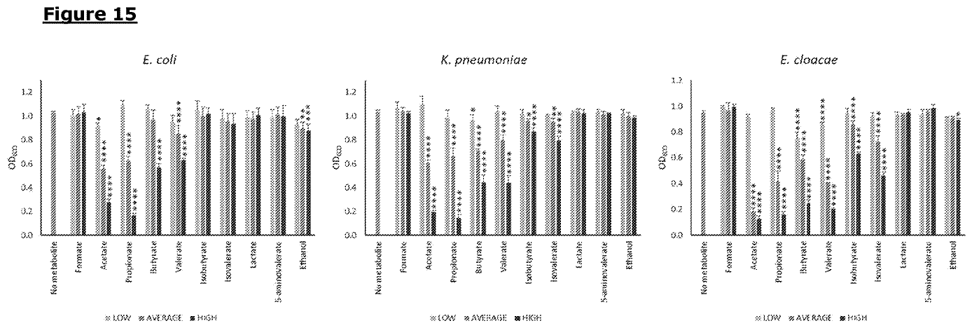

FIG. 14 shows that CRE growth is inhibited by a mixture of metabolites that were found to be decreased in antibiotic treated faecal microbiota. Growth of E. coli, K. pneumoniae, or E. cloacae in LB supplemented with a metabolite mixture or unsupplemented (no metabolites control) at pH 6, 6.5, or 7 after 16 hours of incubation under anaerobic conditions. Growth of each isolate was measured with 6 wells in 2 independent experiments. Unpaired t-test. *=P≤0.05, **=P≤0.01, ***=P≤0.001, ****=P≤0.0001.

FIG. 15 confirms that CRE growth was inhibited by individual metabolites in a dose-dependent manner. Growth of E. coli, K. pneumoniae, or E. cloacae in LB supplemented with an individual metabolite at low, average, or high concentrations (mimicking stool concentrations) or unsupplemented (no metabolite control) at pH 6.5 after 16 hours of incubation under anaerobic conditions. Growth of each isolate was measured with 6 wells in 2 independent experiments. One-way ANOVA followed by Dunn's multiple comparison test (each metabolite was compared to the no metabolite control). *=P≤0.05, **=P≤0.01, ***=P≤0.001, ****=P≤0.0001.

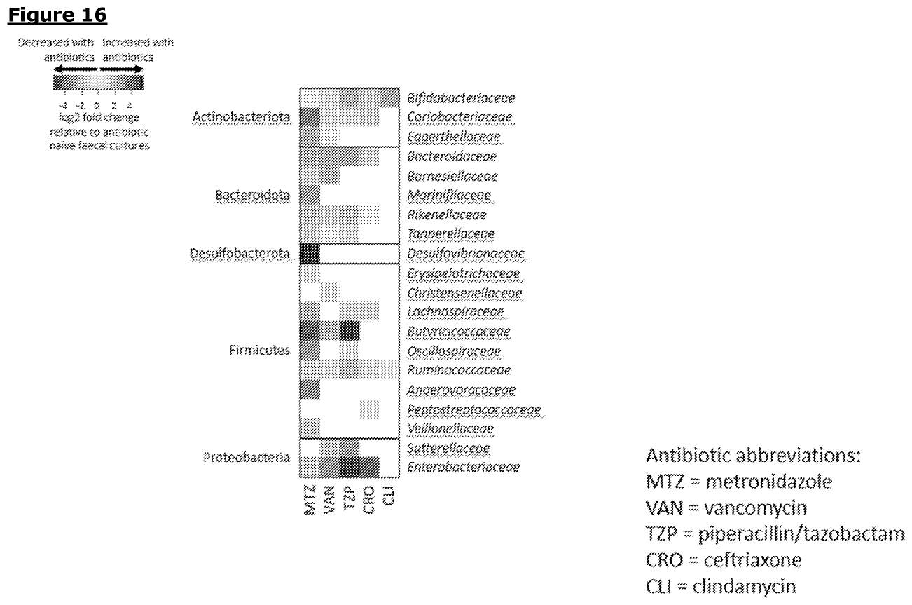

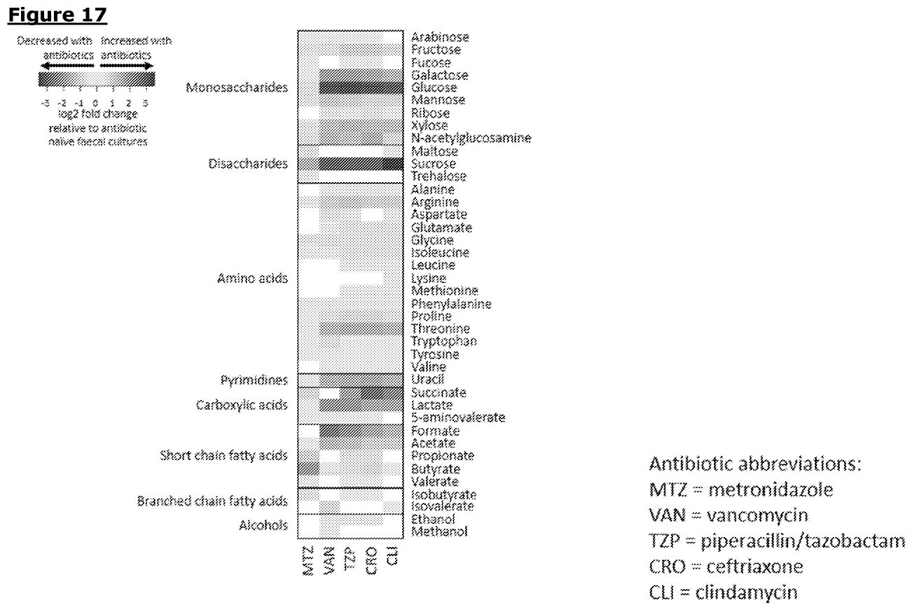

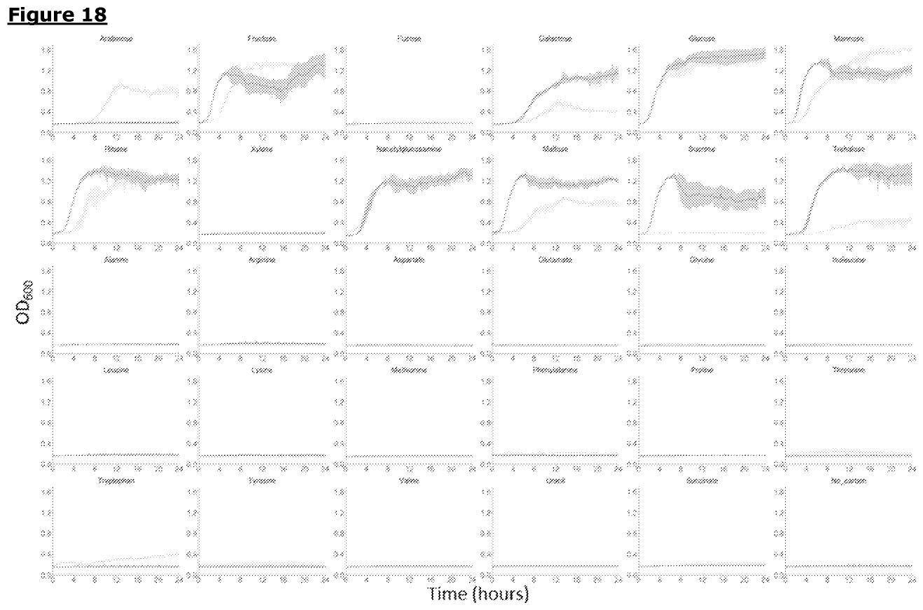

FIG. 16 shows the results of a 16S sequencing heatmap for faecal batch cultures, and the changes in bacterial families in antibiotic-perturbed faecal microbiota. Log 2 fold change in bacterial families significantly decreased with antibiotics (blue) or significantly increased with antibiotics (red), relative to antibiotic-naïve faecal cultures. Bacterial families that were not significantly changed with antibiotics were not plotted (white). Wilcoxon signed rank test with Benjamini Hochberg FDR, p<0.05. The antibiotics tested were antibiotics that are known to promote the intestinal colonisation with VRE.

FIG. 17 shows an NMR heatmap for faecal batch cultures, and the changes in nutrients and metabolites in antibiotic-perturbed faecal microbiota. Log 2 fold change in nutrients and metabolites significantly increased with antibiotics (red) and nutrients and metabolites significantly decreased with antibiotics (blue), relative to antibiotic-naïve faecal cultures. Nutrients and metabolites that were not significantly changed with antibiotics were not plotted (white). Wilcoxon signed rank test with Benjamini Hochberg FDR, p<0.05. The antibiotics tested were antibiotics that are known to promote the intestinal colonisation with VRE.

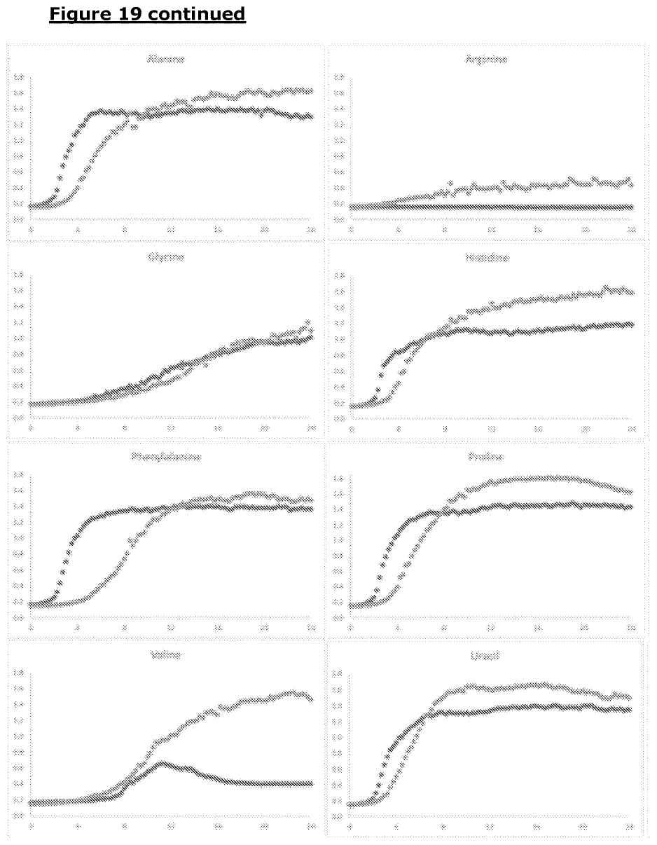

FIG. 18 shows that VRE growth is supported by individual carbon sources found to be elevated after antibiotic treatment. Averaged growth curves for E. faecalis and E. faecium grown on minimal medium supplemented with a single carbon source (or water as the no carbon control) under aerobic conditions. Growth of each isolate was measured with 6 wells in 2-3 independent experiments. The average growth is shown with bold lines and the standard deviation are shown with the shaded bands. Blue=Enterococcus faecalis, green=Enterococcus faecium.

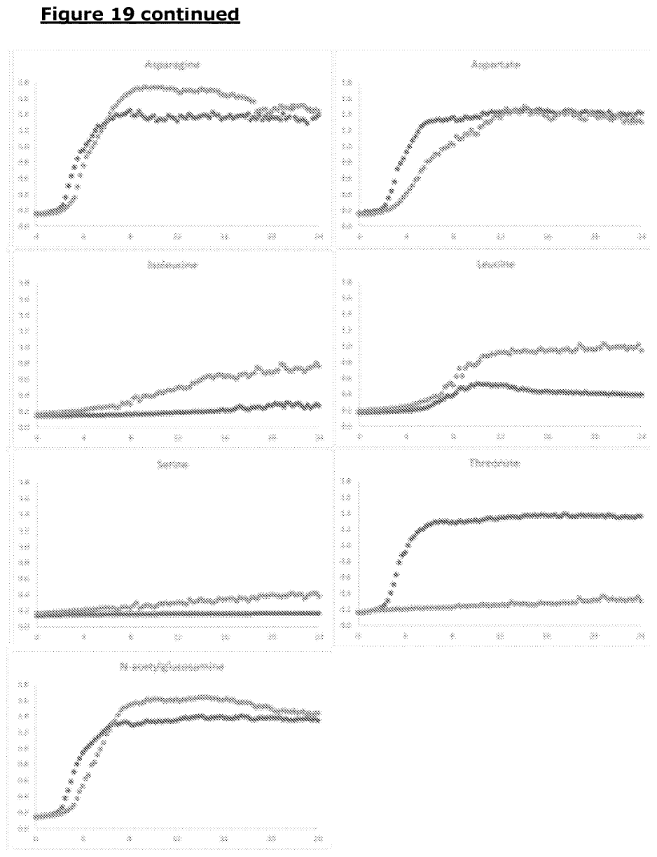

FIG. 19 shows averaged growth curves showing the growth of VRE in a minimal media with a mixture of nitrogen sources elevated after antibiotic treatment. In this leave-one-out design individual nitrogen sources were left out of the mixture to measure their effect on CRE growth. Growth of each isolate was measured with 6 wells in 2-3 independent experiments. The average growth is shown with dotted lines. Blue=Enterococcus faecalis, green=Enterococcus faecium. Growth was performed under aerobic conditions.

FIG. 20 shows that carbapenem-resistant E. coli growth was more inhibited in the presence of a metabolite mixture+commensal combination than in the presence of either the metabolite mixture alone or the commensal mixture alone. Growth of NDM-5 E. coli ST617 in a minimal medium with 1% glucose (pH 6.5) and supplemented with water (positive control), a metabolite mixture (mix of acetate, propionate, butyrate, and valerate), commensal mixture (10 Bacteroides spp., 6 Bifidobacterium spp., and Collinsella aerofaciens), or the metabolite mixture+commensal mixture. One-way ANOVA with Dunn's multiple comparison test (each group was compared to the metabolite mixture+commensal mixture). *=P≤5 0.05, ***=P≤0.001, ****=P≤0.0001. Data are presented as mean values±SD.

### EXAMPLES

The intestine is the primary colonisation site and reservoir for pathogens called carbapenem-resistant Enterobacteriaceae (CRE) and vancomycin-resistant Enterococcus (VRE). CRE and VRE intestinal colonisation precedes the development of other serious antibiotic-resistant infections, such as bloodstream infections or recurrent urinary tract infections. Patients would benefit from the removal of CRE and VRE from their intestines before they go on to develop these serious infections.

The healthy gut is colonised by a diverse collection of microbes collectively referred to as the gut microbiota. A healthy gut microbiota provides protection against intestinal colonisation with CRE and VRE. Nutrients are limited in the intestine and microbes must compete with each other to use these nutrients to support their growth. A healthy gut microbiota outcompetes CRE and VRE for these nutrients and also produces compounds called metabolites that can inhibit CRE and potentially VRE growth. However, antibiotic-mediated killing of gut microbiota members allows CRE and VRE to colonise and dominate the intestine. Antibiotics increase the concentration of nutrients available to support CRE and VRE growth coupled with a reduction in the concentration of metabolites that can inhibit CRE and VRE growth.

As discussed in the examples below, the inventors' aims were to develop new microbiome therapeutics consisting of a synthetic microbial consortium (specific members of the gut microbiota) and inhibitory metabolites. The inhibitory metabolites will cause an initial reduction in CRE or VRE growth. The synthetic microbial consortium will then colonise the intestine, outcompete CRE or VRE for nutrients, and further inhibit CRE or VRE growth by producing inhibitory metabolites, resulting in a long-term reduction in CRE or VRE growth.

### Materials and Methods for Carbapenem-Resistant Enterobacteriaceae (CRE) Experiments

### Carbapenem-Resistant Enterobacteriaceae Isolates

New Delhi Metallo-β-lactamase 1 (NDM-1) Escherichia coli, NDM-1 Klebsiella pneumoniae, and NDM-1 Enterobacter cloacae were isolated from patients with intestinal CRE colonisation from the Imperial College Healthcare NHS Trust via rectal swab (REC ref. 19/LO/0112). The species were assigned by matrix assisted laser desorption ionization-time of flight (MALDI-TOF) mass spectrometry and later confirmed with 16S rRNA gene sequencing. CRE status was determined using X-pert Carba-R PCR detection (Cepheid, Sunnyvale, CA, USA), (Otter, J. A. et al. Detecting carbapenemase-producing Enterobacterales (CPE): an evaluation of an enhanced CPE infection control and screening programme in acute care. J Antimicrob Chemother 75, 2670-2676 (2020)).

### Faecal Batch Culture Experiments

Ex vivo faecal batch culture experiments were performed to investigate the effects of antibiotics on the faecal microbiota. Faecal microbiota cultured in these experiments are highly reproducible, allowing for the parallel testing of antibiotic-treated and antibiotic-naïve control groups that are inoculated from a single faecal sample (Ladirat, S. E. et al. High-throughput analysis of the impact of antibiotics on the human intestinal microbiota composition. J Microbiol Methods 92, 387-97 (2013)). Comparison of antibiotic-naïve vs antibiotic-treated faecal microbiota is extremely challenging in human studies due to the difficulties collecting samples from patients prior to the start of antibiotic treatment. These faecal batch culture experiments also allow us to test the faecal microbiota using a standardised growth medium mimicking nutrients found in the distal gut (McDonald, J. A. et al. Evaluation of microbial community reproducibility, stability and composition in a human distal gut chemostat model. J. Microbiol. Methods 95, 167-174 (2013)). Studies of nutrient availability and metabolite production can be challenging to perform in humans as there may be differences in host diet between healthy volunteers and antibiotic-treated patients. Finally, these experiments allow us to investigate the impacts of antibiotics on a human gut microbiota. It is well known that there are significant differences between human and mouse faecal microbiota, and that important human-specific genera are completely absent in mice (Nguyen, T. L., Vieira-Silva, S., Liston, A. & Raes, J. How informative is the mouse for human gut microbiota research? Dis. Model. Mech. 8, 1-16 (2015). Krych, L., Hansen, C. H., Hansen, A. K., van den Berg, F. W. & Nielsen, D. S. Quantitatively different, yet qualitatively alike: a meta-analysis of the mouse core gut microbiome with a view towards the human gut microbiome. PLoS One 8, e62578 (2013)).

Ethical approval was received by the London—Queen Square Research Ethics Committee (REC ref. 19/LO/0112) and the South Central—Oxford C Research Ethics Committee (REC ref. 16/SC/0021 and 20/SC/0389). Faecal donors did not receive antibiotic treatment in the 6+ months prior to donation.

The inventors performed faecal culture experiments to measure the effects of eight broad-spectrum antibiotics on the faecal microbiota collected from 11 healthy human donors. Faecal culture experiments were performed under anaerobic conditions using a complex gut growth medium that was designed to mimic the nutrients found in the human distal gut (McDonald, J. A. et al. Evaluation of microbial community reproducibility, stability and composition in a human distal gut chemostat model. J. Microbiol. Methods 95, 167-174 (2013)). For each donor, fresh faeces were inoculated into a gut growth medium at a 2% (w/v) concentration that had been supplemented with one of the following antibiotics (at faecal concentrations): 2 μg/ml meropenem (MEM), 2 μg/ml imipenem/cilastatin (IPM), 37 μg/ml ertapenem (ETP), 139 μg/ml piperacillin/tazobactam (TZP), 152 μg/ml ceftriaxone (CRO), 22 μg/ml ceftazidime (CAZ), 2 μg/ml cefotaxime (CTX), 139 μg/ml ciprofloxacin (CIP), or water (Ladirat, S. E. et a/. High-throughput analysis of the impact of antibiotics on the human intestinal microbiota composition. J Microbiol Methods 92, 387-97 (2013). Moon, Y. S., Chung, K. C. & Gill, M. A. Pharmacokinetics of meropenem in animals, healthy volunteers, and patients. Clin Infect Dis 24, S249-S255 (1997). Kager, L., Brismar, B., Malmborg, A. S. & Nord, C. E. Imipenem concentrations in colorectal surgery and impact on the colonic microflora. Antimicrob Agents Chemother 33, 204-8 (1989). Pletz, M. et al. Ertapenem pharmacokinetics and impact on intestinal microflora, in comparison to those of ceftriaxone, after multiple dosing in male and female volunteers. Antimicrob Agents Chemother 48, 3765-72 (2004). Nord, C. E., Brismar, B., Kasholm-Tengve, B. & Tunevall, G. Effect of piperacillin/tazobactam treatment on human bowel microflora. J Antimicrob Chemother 31, 61-5 (1993). Burdet, C. et al. Ceftriaxone and Cefotaxime Have Similar Effects on the Intestinal Microbiota in Human Volunteers Treated by Standard-Dose Regimens. Antimicrob Agents Chemother 63, 2244 (2019). Pessôa de Menezes e Silva, C H. Elaboration and evaluation of a new screening medium for detection and presumptive identification of extended-spectrum beta-lactamase-producing organisms (ESBL). Braz J Microbiol 31, 271-274 (2000). Brismar, B., Edlund, C., Malmborg, A. S. & Nord, C. E. Ciprofloxacin concentrations and impact of the colon microflora in patients undergoing colorectal surgery. Antimicrob Agents Chemother 34, 481-3 (1990)). Cultures were incubated anaerobically at 37° C. for 24 hours. Samples were analysed by ¹H-NMR spectroscopy (for nutrient and metabolite profiling) and 165 rRNA gene sequencing with 16S rRNA gene qPCR (for bacterial composition and biomass) (McDonald, J. A. K. et a/. Inhibiting Growth of Clostridioides difficile by Restoring Valerate, Produced by the Intestinal Microbiota. Gastroenterology 155, 1495-1507 (2018)).

16S rRNA Gene Sequencing and 16S rRNA Gene qPCR

DNA was extracted from 250 μl of faecal batch culture sample using the DNeasy PowerLyzer PowerSoil Kit (Qiagen, Germany) according to manufacturer's instructions, with the addition of a bead beating step for 3 minutes at speed 8 in a Fast-Prep 24 bead beater (MP Biomedicals, USA). DNA was stored at −80° C. until it was ready to be used.

Illumina's 16S metagenomic sequencing library preparation protocol was used to generate sample libraries amplifying the V1-V2 regions of the 16S rRNA gene, as previously described (Mullish, B. H. et al. Functional microbiomics: Evaluation of gut microbiota-bile acid metabolism interactions in health and disease. Methods 149, 49-58 (2018)). The SequalPrep Normalization Plate Kit (Life Technologies, UK) was used to clean up and normalise the index PCR reactions. The NEBNext Library Quant Kit for Illumina (New England Biolabs, UK) was used to quantify sample libraries. Sample libraries were sequenced on an Illumina MiSeq platform (Illumina Inc., UK) using the MiSeq Reagent Kit v3 (Illumina) and paired-end 300 bp chemistry. 16S rRNA gene qPCR was performed to quantify the bacterial biomass of each sample as previously described (McDonald, J. A. K. et al. Inhibiting Growth of Clostridioides difficile by Restoring Valerate, Produced by the Intestinal Microbiota. Gastroenterology 155, 1495-1507 (2018)).

16S rRNA gene sequencing data were imported into R and processed using the standard DADA2 pipeline (version 1.18.0) (Callahan, B. J. et al. DADA2: High-resolution sample inference from Illumina amplicon data. Nat Methods 13, 581-3 (2016)). The SILVA bacterial database version 138.1 was used to classify the sequence variants. 16S rRNA gene sequencing data was expressed as absolute abundances using the 16S rRNA gene qPCR data, as previously described (McDonald, J. A. K. et al. Inhibiting Growth of Clostridioides difficile by Restoring Valerate, Produced by the Intestinal Microbiota. Gastroenterology 155, 1495-1507 (2018)). 16S rRNA gene sequencing data (weighted as absolute abundances) was analysed using Wilcoxon signed rank test with Benjamini & Hochberg FDR correction using the DA.wil function within in the DAtest R package version 2.7.18.

### ¹H-NMR Spectroscopy

Supernatants were prepared from faeces by vortexing 300 mg of faeces in 900 μl of HPLC-grade water for 10 minutes at 3000 rpm followed by centrifugation at 17,000×g for 10 minutes at 4° C. Supernatants were prepared from faecal culture samples by centrifugation at 17,000×g for 10 minutes at 4° C. Supernatants were frozen at −80° C. until they were ready for analysis.

¹H-NMR spectroscopy was performed as previously described (McDonald, J. A. K. et al. Inhibiting Growth of Clostridioides difficile by Restoring Valerate, Produced by the Intestinal Microbiota. Gastroenterology 155, 1495-1507 (2018)). Briefly, sample supernatants were randomized and defrosted at room temperature for 1 hour. Sample supernatants were then centrifuged for 10 min at 17 000×g and 4° C. The pellet was discarded and 400 μl of supernatant was added to 250 μl NMR buffer (28.85 g Na2HPO4, 5.25 g NaH2PO4, 1 mM TSP, 3 mM NaN3, deuterium oxide to 1 L, pH 7.4). 600 μl of sample was used to fill each 5 mm NMR tube to be loaded into the SampleJet system.

¹H-NMR spectroscopy was performed using the Bruker AVANCE III HD 800 MHz spectrometer (Bruker Bio-Spin, Rheinstetten, Germany). 1D spectra (a standard NOESYGPPR1D pulse sequence (RD-90°-t1-90°-tm-90°-ACQ)) as well as 2D spectra (standard 2D JRESGPPRQF pulse sequence) were acquired. A recycle delay of 4 seconds and mixing time of 100 ms was used. The 90° pulse length was approximately 10 μs and 32 scans were recorded.

NMR spectra were imported into MATLAB r2019b (The Mathworks, USA) using an in-house script. Spectra were aligned and the water (4.6-5 ppm) and TSP (−0.2-0.2 ppm) peaks were cut. Compounds were identified by comparison to spectral databases in Chenomx NMR Suite (Chenomx, Canada). Representative peaks were integrated for quantification of compounds. Peak integration values from the 1H-NMR data were analysed using Wilcoxon signed rank test with Benjamini & Hochberg FDR correction using the DA.wil function within in the DAtest R package version 2.7.18.

mixOmics

Regularised canonical correlation analysis (rCCA) was used to correlate 16S rRNA gene sequencing data (family level) with 1H-NMR data using the mixOmics library within R, as previously described (McDonald, J. A. K. et al. Inhibiting Growth of Clostridioides difficile by Restoring Valerate, Produced by the Intestinal Microbiota. Gastroenterology 155, 1495-1507 (2018). K. Le Cao, F. Rohart, I. Gonzalez and S. Dejean. mixOmics: Omics Data Integration Project. R package version 6.1.2. https://CRAN.R-project.org/package=mixOmics (2017)). In unit representation plots each point represents a single faecal culture sample that was projected into the XY-variate space. In the correlation circle plots variables projected in the same direction from the origin have a positive correlation, and variables projected in opposite directions form the origin have a negative correlation. Variables sitting at farther distances from the origin have stronger correlations than variables sitting closer to the origin.

### Carbon and Nitrogen Utilisation Assays

Growth of CRE isolates on sole carbon or nitrogen sources was tested by inoculating each CRE isolate into a minimal medium supplemented with the nutrient of interest. First, CRE isolates were passaged from frozen glycerol stocks on Reasoner's 2A agar (R2A) plates. Cells were then suspended in M9 minimal media to a turbidity of 42% using a Turbidimeter (Biolog Inc.), then diluted a further 1 in 6 to inoculate the carbon or nitrogen utilisation assays. M9 minimal media contained the following (at final concentrations): 22 mM KH₂PO₄, 42 mM Na2HPO4, 9 mM NaCl, 0.49 mM MgSO₄·7H2O, 0.09 mM CaCl₂), 0.011 mM FeSO₄·7H2O. For the carbon assays the M9 minimal medium was also supplemented with 0.1% (w/v) NH₄Cl (as the nitrogen source) and the carbon sources were individually tested at 0.5% (w/v) concentration. For the nitrogen assays the M9 minimal medium was also supplemented with 0.5% (w/v) glucose (as the carbon source) and the nitrogen sources were individually tested at 0.1% (w/v) concentration. Cultures were incubated anaerobically and aerobically in a plate reader and OD600 readings were collected every 15 minutes for 24 hours.

### Nutrient Preference Assays

CRE isolates were passaged once from frozen glycerol stocks on R2A plates. Cells were suspended in M9 minimal media to a turbidity of 42% using a Turbidimeter (Biolog Inc.), then diluted 1 in 6 to inoculate the nutrient preference assays. M9 minimal media was supplemented with 0.015% of each of the following: L-arabinose, D-fructose, L-fucose, D-galactose, D-glucose, D-mannose, D-ribose, D-xylose, N-acetylglucosamine, D-maltose, sucrose, D-trehalose, L-alanine, L-arginine, L-aspartate, L-glutamate, L-glycine, L-isoleucine, L-leucine, L-lysine, L-methionine, L-phenylalanine, L-proline, L-threonine, L-tryptophan, L-tyrosine, L-valine, uracil, and succinate. Cultures were incubated anaerobically and aerobically in a plate reader and OD600 readings were collected every 15 minutes for 24 hours. Cultures were also incubated anaerobically and aerobically for 24 h and samples were collected at 0, 4, 8, and 24 hours post-inoculation for 1H-NMR spectroscopy. Changes in nutrients and metabolites by CRE isolates was compared to the sterile media control over time using a two-way mixed ANOVA followed by pairwise comparisons with Bonferroni correction.

### AMiGA

Growth curves were analysed in Python (v3.6.5) using the AMiGA software (Midani, F. S., Collins, J. & Britton, R. A. AMiGA: Software for Automated Analysis of Microbial Growth Assays. mSystems 6, e0050821 (2021)). Growth of each isolate was measured with 6 wells from 2-3 independent experiments. Growth curves were normalised to the no substrate control using the subtraction method. AMiGA performed Gaussian Process regression to test differential growth between the substrate and the no substrate control. The functional difference in the OD (and its credible interval) was also computed between the substrate and the no substrate control.

Metabolite Measurements from Healthy Faeces

The concentration of 10 microbial metabolites were measured from the faeces of 12 healthy donors by 1H-NMR spectroscopy. Table 1 shows the approximate average, minimum, and maximum concentration of each metabolite that was measured in these faecal samples, and these concentrations were tested in the metabolite inhibition assays.

TABLE 1
Concentration of metabolites measured
in human faeces from 12 healthy donors

| | Lowest | Average | Highest |
|---|---|---|---|
| | concentration | concentration | concentration |
| Metabolite | (mM) | (mM) | (mM) |
| Formate | 0.05 | 0.10 | 0.15 |
| Acetate | 10 | 65 | 120 |
| Propionate | 5 | 15 | 35 |
| Butyrate | 3 | 15 | 40 |
| Valerate | 0.5 | 3.5 | 12 |
| Isobutyrate | 0.5 | 2 | 5 |
| Isovalerate | 0.5 | 2 | 5 |
| Lactate | 0.3 | 0.7 | 1.2 |
| 5-aminovalerate | 0.3 | 1 | 4 |
| Ethanol | 0.6 | 10 | 60 |

### Mixed Metabolite Inhibition Assay

A mixture of the metabolites from Table 1 were tested in an inhibition assay against each CRE isolate. The metabolite mixture tested each metabolite at a concentration mimicking their average concentration in healthy faeces (from Table 1). LB was supplemented with the metabolite mixture and compared to a no metabolite control. The assay was run at pH 6.0, 6.5, and 7.0 to mimic the pH range found in healthy faeces (Payne, A. N., Zihler, A., Chassard, C. & Lacroix, C. Advances and perspectives in in vitro human gut fermentation modeling. Trends Biotechnol. 30, 17-25 (2012). Shelton, C. D. et al. Salmonella enterica serovar Typhimurium uses anaerobic respiration to overcome propionate-mediated colonization resistance. Cell Rep 38, 110180 (2022)). Overnight cultures of each CRE isolate were inoculated at 103 CFU/ml. Cultures were incubated anaerobically for 16 hours at 37° C. Growth was measured at OD600 after 16 hours of incubation. Growth of the CRE isolate with the metabolite mixture was compared to growth without the metabolite mixture at each pH using an unpaired t-test.

### Individual Metabolite Inhibition Assay

Each metabolite was tested individually in an inhibition assay against each CRE isolate. Each metabolite was tested at three concentrations: the low, average, and high concentrations that were measured in healthy faeces (from Table 1). LB was supplemented with each metabolite (at the three different concentrations) and compared to a no metabolite control. The assay was run at pH 6.5 as this was representative of the distal gut and also showed inhibition in the mixed metabolite assay. Overnight cultures of each CRE isolate were inoculated at 103 CFU/ml. Cultures were incubated anaerobically for 16 hours at 37° C. Growth was measured at OD600 after 16 hours of incubation. Growth of the CRE isolate in the presence of each metabolite was compared to the no metabolite control using a one-way ANOVA followed by Dunn's multiple comparison test.

Carbapenem Resistant Escherichia coli

E. coli ST617 (NDM-5) was isolated from patients with intestinal CRE colonisation from the Imperial College Healthcare NHS Trust (London, UK) via rectal swab (with approval from the London-Queen Square Research Ethics Committee, 19/LO/0112).

### Commensal Gut Bacteria

Phocaeicola vulgatus, Collinsella aerofaciens, Bifidobacterium longum, Phocaeicola dorei, Bacteroides caccae, Bacteroides xylanisolvens, Bacteroides uniformis, Bacteroides ovatus, and Bacteroides stercoris were isolated from healthy human donors with ethical approval from the South Central-Oxford C Research Ethics Committee (20/SC/0389). Bacteroides intestinalis (DSM 108646) and Phocaeicola plebeius (DSM 17135) were purchased from DSMZ Bacteria collection. Bifidobacterium adolescentis (LMG 29394), Bifidobacterium catenulatum (LMG 11043), Bifidobacterium pseudocatenulatum (LMG 29397), Bifidobacteroium breve (LMG 11040), and Bifidobacterium dentium (LMG 10507) were purchased from BCCM/LMG Bacteria Collection.

### Minimal Media

A Minimal Medium (MM) was developed to support the growth of the 16 commensal strains used in this work. The media contains the following: potassium phosphate dibasic (9.4 g/L), potassium phosphate monobasic (4.2 g/L), ammonium sulphate (1.126 g/L), L-cysteine hydrochloride (1 g/L), yeast extract (1 g/L), sodium chloride (0.89 g/L), calcium chloride (81 mg/L), magnesium sulphate (30.3 mg/L), magnesium chloride (24 mg/L), ethylenediaminetetraacetic acid (5 mg/L), manganese (II) chloride (5 mg/L), pantetheine (5 mg/L), nicotinic acid (5 mg/L), hemin (3.75 mg/L), L-arginine (2.5 mg/L), L-asparagine (2.5 mg/L), L-aspartate (2.5 mg/L), L-glutamine (2.5 mg/L), L-glycine (2.5 mg/L), L-histidine (2.5 mg/L), L-isoleucine (2.5 mg/L), L-leucine (2.5 mg/L), L-lysine HCl (2.5 mg/L), L-methionine (2.5 mg/L), L-phenylalanine (2.5 mg/L), L-serine (2.5 mg/L), L-threonine (2.5 mg/L), L-tryptophan (2.5 mg/L), L-tyrosine (2.5 mg/L), L-valine (2.5 mg/L), iron sulphate (2 mg/L), zinc chloride (1 mg/L), adenosine (1 mg/L), thymine (1 mg/L), uracil (1 mg/L), guanosine (1 mg/L), menadione (0.375 mg/L), nickel dichloride (0.2 mg/L), cobalt (II) chloride (0.2 mg/L), copper (II) chloride (0.1 mg/L), boric acid (0.1 mg/L), Kao and Michayluk vitamin solution [Sigma, K3129, 50× Stock](15 ml/L), and Tween 80(0.1 mL/L). The media was prepared in deionised water and sterilized with a 0.2-micron filter. The minimal media was prepared with a final glucose concentration of 1% and adjusted to pH 6.5.

### Preparation of Commensal Gut Species

The commensal gut bacteria were plated onto Fastidious Anaerobe Agar containing 5% horse blood and were incubated for 24 hours at 37° C. under anaerobic conditions in a Whitley DG250 Anaerobic Workstation. For each of the commensal gut bacteria, one colony was inoculated into 1 ml of MM with 1% glucose and incubated for 6 hours at 37° C. under anaerobic conditions. To prepare the “commensal gut bacteria mix”, an equal volume of each species were combined and were diluted 1:20 into fresh MM with 1% glucose.

### Preparation of Inhibitory Metabolites

The concentration of inhibitory metabolites (acetate, valerate, butyrate, and propionate) was measured in human faeces from 12 healthy donors as previously reported¹. The average concentration of these metabolites was used acetate (65 mM), valerate (3.5 mM), butyrate (15 mM), and propionate (15 mM), and adjusted to pH 6.5 to reflect the pH of the healthy gut^{2,3}.

### Inhibition Assay

The purpose of this experiment was to determine whether a combination of commensal gut bacteria and microbial metabolites is more inhibitory to E. coli ST617 than either agent alone. Four conditions were used 1=E. coli ST617 alone, 2=E. coli ST617+metabolite mix, 3=E. coli ST617+gut commensal mix, 4=E. coli ST617+metabolite mix+gut commensal mix. Into each of the four conditions, E. coli ST617 was added at 1×103 CFU/ml. All conditions were incubated for 24 hours at 37° C. under anaerobic conditions, then plated on Luria broth agar under aerobic conditions to quantify E. coli ST617 growth (the aerobic conditions would prevent the growth of the anaerobic bacteria within the gut commensal mix).

Materials & Methods for vancomycin-resistant Enterococcus (VRE) Experiments

### Faecal Batch Culture

### Donor Involvement

Healthy donors provided faecal samples within the ethical guidelines of the Imperial Hepatology and Gastroenterology Biobank (REC Reference No. 20/SC/0389, approved by the South Central—Oxford C Research Ethics Committee on 12 Feb. 2021). Recruited donors were verbally assessed and informed of study objectives prior to completing case report and consent forms. All participation was voluntary and within Biobank ethical guidelines. Participants were considered if they were over 18 years of age and had not received antibiotics in six months prior to donation. Participants were pre-screened to rule out digestive irregularities or family history of gastrointestinal disease. Of the 15 donors, 6 were female and 9 were male with an age distribution of 30 f 26. Two donors were vegetarian, two were pescatarian and the remaining 11 consumed a western diet.

### Sample Collection and Batch Culture Preparation

Samples, collected in sterile stool containers, were immediately transferred to an anaerobic chamber. For every sample, aliquots of faeces were either immediately stored at −80° C. or prepared for nuclear magnetic resonance (NMR) spectroscopy by vortexing for 10 minutes with HPLC-grade water to create a 25% faecal slurry. This was then centrifuged (4° C., 17,000×g) for 10 minutes before the supernatant was extracted and stored at −80° C.

Using stool samples from the 12 donors, faecal cultures were treated with either water or antibiotics in triplicate. The 20% was prepared using faeces and 0.9% degassed saline in a stomacher strainer bag homogenized in a Seward Stomacher Blender for one minute at normal speed. Cultures were prepared with stool to a final concentration of 2% and either water, vancomycin (500 μg/mL), metronidazole (25 μg/mL), ceftriaxone (152 μg/mL), piperacillin/tazobactam (139 μg/mL), ampicillin (105 μg/mL), or clindamycin (383 μg/mL). Cultures were incubated anaerobically for 24 hours at 37° C.

DNA Isolation & 165 rRNA Gene Sequencing

DNA extraction was performed with the PowerLyzer PowerSoil DNA isolation kit (QIAGEN, Cat. No. 12855-100) using the manufacture protocol. The cell lysis protocol was altered to using a bead beater for three 1-minute cycles with 1-minute rests between rounds using the FastPrep 24 (SKU 116004500) machine. DNA concentrations were confirmed Qubit dsDNA BR Assay Kit (Cat. No. Q32850) and a Qubit fluorometer (Cat. No. Q32866). Samples were diluted to 5 ng/μl prior to library preparation and 16S rRNA gene sequencing. To amplify the V1-V2 region of the 15S rRNA gene, sample libraries were prepared using previously determined methods (Mullish, B. H., et al., Functional microbiomics: Evaluation of gut microbiota-bile acid metabolism interactions in health and disease. Methods, 2018. 149: p. 49-58). Sequencing was performed with the Illumina MiSeq platform using paired end 300 bp reads (Illumina Incorporated).

### ¹H-NMR Spectroscopy

NMR buffer was prepared with sodium phosphate monobasic (5.25 g/L), sodium phosphate dibasic (28.85 g/L), 3-trimethylsilyl propionic acid (1 mM), and sodium azide (3 mM) in deuterium oxide. The supernatant was extracted from thawed samples, mixed with the buffer and loaded into 5 mm NMR sample tubes. NMR spectra were acquired at 300 K using a Bruker AVANCE III 800 MHz NMR spectrometer. The pulse sequence was set as follows: pulse sequence (RD-90°-t1-90°-tm-90°-ACQ) with a recycle delay of 4 seconds and a mixing time of 100 ms. For each spectrum, 32 scans were collected. Spectra were imported into MATLAB R2020b where the water and TSP peaks were removed, and manual alignment was conducted. Spectra were normalised with “JTPnormalise” script.

### Preparation of VRE Minimal Media

VRE minimal media consists of potassium phosphate monobasic (4 g/L), potassium phosphate dibasic (14 g/L), sodium citrate (1 g/L), magnesium sulphate (0.2 g/L), ammonium sulphate (2 g/L), adenosine (0.04 g/L), thymine (0.04 g/L), Kao and Michayluk vitamin solution [Sigma, K3129, 50× Stock](5×), glucose (1 g/L) and amino acid solution [Sigma, R7131, RPMI-1640, 50×](25×). If the study was performed under anaerobic conditions, fumarate (5.2 g/L) was added to increase growth of VRE strains. Carbon-free minimal media was prepared by leaving out glucose while nitrogen-free minimal media was prepared without the amino acid solution. Media was filter sterilized with a 0.22 micron filter and used immediately aerobically or degassed overnight if used anaerobically.

### Nutrient Utilization Protocol

VRE strains were grown on R2A overnight before the cells were suspended in 0.9% saline to an OD of 0.38 (λ=595 nm). A 10% dilution of the cell suspension was prepared using VRE minimal media to achieve a final well OD of 0.6 (λ=595 nm) greater than the sterile media OD. Both carbon and nitrogen source utilization were evaluated by adding the inoculated media and a filter-sterilised nutrient source solution into a well in a 1:1 ration to a final volume of 100 μL in aerobic tests and 200 μL in anaerobic tests. Carbon sources were tested individually and compared to a carbon-free control. To test nitrogen source utilization, a nitrogen source solution was prepared containing L-alanine (2 g/L), L-arginine (2.5 g/L, L-asparagine (0.71 g/L), L-aspartate (0.25 g/L), L-cysteine (0.625 g/L), L-glutamate (0.25 g/L), L-Glycine (0.125 g/L), L-histidine (0.1875 g/L), L-isoleucine (0.625 g/L), L-leucine (0.625 g/L), L-lysine HCl (0.5 g/L), L-methionine (0.1875 g/L), L-phenylalanine (0.1875 g/L), L-proline (0.25 g/L), L-serine (0.375 g/L), L-threonine (0.25 g/L), L-tryptophan (0.0625 g/L), L-tyrosine (0.29 g/L), L-valine (0.25 g/L), N-acetylglucosamine (2 g/L), and uracil (2.5 g/L). To test the strains' ability to utilize individual nitrogen sources, separate solutions were prepared eliminating one nitrogen source each. The change in growth compared to the complete nitrogen source solution control was used to determine nitrogen utilization. All carbon and nitrogen sources were tested in a technical triplicate for each biological replicate. Aerobic plates were incubated with a plate seal while anaerobic plates were not. The aerobic plate reader was set to read the plate (λ=595 nm, 37° C., 22 flashes per well, orbital shaking of 100 rpm for 15 s prior to each read) every 15 minutes for 24 hours. The anaerobic plate reader read the plate (λ=595 nm, 37° C., stationary) every 15 minutes for 24 hours.

### Results

### Example 1—Antibiotic Treatment Decreased the Abundance of Important Bacterial Families in Faecal Microbiota (CRE)

The inventors used antibiotics to study how the loss or reduction of gut commensals from healthy gut microbiota impacted nutrient availability and metabolite production. The inventors performed ex vivo faecal culture experiments to measure the effects of eight broad-spectrum antibiotics (known to promote the intestinal colonisation with CRE) on faecal microbiota from 11 healthy donors. Antibiotics tested included carbapenems (MEM, IPM, ETP), a penicillin/β-lactamase inhibitor (TZP), a fluoroquinolone (CIP), and cephalosporins (CRO, CAZ, CTX). For each experiment a faecal sample was inoculated into a distal gut growth medium supplemented with one of the eight antibiotics or not supplemented with antibiotics (antibiotic-naïve control), and incubated anaerobically for 24 hours.

First, the inventors aimed to determine whether antibiotics that promote CRE intestinal colonisation resulted in the loss or reduction of similar bacterial taxa across the different antibiotics. The inventors measured changes in bacterial taxa in the faecal cultures in response to antibiotics using 16S rRNA gene sequencing (to measure bacterial composition) and 16S rRNA qPCR (to measure bacterial biomass). The inventors found that antibiotics decreased the abundance of several bacterial families, as shown in FIG. 1. Carbapenems (IPM, MEM, ETP) decreased the abundance of families belonging to the phyla Actinobacteriota (Bifidobacteriaceae and Coriobacteriaceae), Bacteroidota (Bacteroidaceae, Barnesiellaceae, Prevotellaceae, Rikenellaceae, Tannerellaceae) and Firmicutes (Erysipelatotrichaceae, Lachnospiraceae, Butyricicoccaceae, Acidaminococcaceae, and Veillonellaceae). TZP (a penicillin/β-lactamase inhibitor) decreased the abundance of Bifidobacteriaceae and Bacteroidaceae. CIP (a fluoroquinolone) decreased the abundance of Bifidobacteriaceae, Barnesiellaceae, and Sutterellaceae. Cephalosporins (CRO, CAZ, CTX) decreased the abundance of Bifidobacteriaceae. Therefore, antibiotic treatment resulted in a decrease in the abundance of important bacterial families in faecal cultures.

### Example 2—Antibiotic Treatment Increased the Concentration of Nutrients and Decreased the Concentration of Microbial Metabolites in Faecal Microbiota (CRE)

The nutrient-niche hypothesis predicts that antibiotic-mediated disruption of the gut microbiota leads to an increase in nutrient availability in the gut, promoting Enterobacteriaceae growth. However, the nutrients that may be impacted by antibiotics have not been described. Moreover, microbial metabolites can be inhibitory to pathogen growth, but antibiotics have also been shown to decrease the concentration of microbial metabolites. Therefore, the inventors next measured the effects of antibiotics on nutrient availability and metabolite production in the antibiotic-treated and antibiotic-naïve faecal cultures using ¹H-NMR spectroscopy.

Antibiotic treatment of faecal cultures increased the concentration of several nutrients, including monosaccharides, disaccharides, and amino acids (FIG. 2). Monosaccharides that increased with antibiotics included 6-carbon sugars (glucose, fructose, galactose, mannose, fucose), 5-carbon sugars (arabinose, ribose, xylose) and N-acetylglucosamine. Disaccharides that increased with antibiotics included maltose, sucrose, and trehalose. Amino acids that increased with antibiotics included alanine, arginine, aspartate, glutamate, glycine, isoleucine, leucine, lysine, methionine, phenylalanine, proline, threonine, tryptophan, tyrosine, and valine. Antibiotics also increased the concentration of uracil (a pyrimidine) and succinate (a carboxylic acid). Therefore, antibiotic treatment of a faecal microbiota resulted in an increase in the amount of nutrients that are available to support the growth of CRE.

Antibiotics also decreased the concentration of metabolites produced by the gut microbiota, including short chain fatty acids (SCFAs), branched chain fatty acids (BCFAs), carboxylic acids, and ethanol (FIG. 2). Short chain fatty acids that decreased included formate, acetate, propionate, and valerate. Butyrate decreased in ETP, TZP, CIP, and CRO-treated faecal cultures but increased in MEM, IPM, CAZ, and CTX-treated cultures. Branched chain fatty acids that decreased with antibiotics included isobutyrate and isovalerate. Carboxylic acids that decreased with antibiotics included lactate and 5-aminovalerate. Therefore, antibiotic treatment resulted in a decrease in the concentration of microbial metabolites which may be important for maintaining colonisation resistance against CRE.

### Example 3—Bacterial Taxa that Decreased with Antibiotics were Positively Correlated with Metabolites and Negatively Correlated with Nutrients that were Changed with Antibiotic Treatment (CRE)

Next, the inventors used rCCA modelling of the antibiotic-treated faecal culture data to measure correlations between bacterial families and nutrients/metabolites that correspond to antibiotic treatment. The aim of this analysis was to link changes in bacteria families following antibiotics to the enrichment of nutrients and reduction in metabolites, to provide targets for the future development of microbiome therapeutics to restore colonisation resistance and inhibit CRE growth.

The unit representation plot showed a separation between antibiotic-treated and antibiotic-naïve faecal cultures along the first canonical variate, indicating a separation in these groups based on their bacterial taxa and nutrient/metabolite profiles (FIG. 3). The correlation circle plot showed that the separation between antibiotic-treated and antibiotic-naïve faecal cultures was due to an increase in monosaccharides, disaccharides, amino acids, uracil, and succinate and a decrease in SCFAs, BCFAs, 5-aminovalerate and ethanol in antibiotic-treated cultures (FIG. 3). This plot also showed strong negative correlations between bacterial families that were decreased with antibiotics and monosaccharides, disaccharides, amino acids, uracil, and succinate. It also showed strong positive correlations between bacterial families that were decreased with antibiotics and SCFAs, BCFAs, 5-aminovalerate, and ethanol.

Therefore, antibiotic treatment using antibiotics that promote CRE intestinal colonisation resulted in a reduction of bacterial families that are positively correlated with metabolites (indicating that they may produce these metabolites) and negatively associated with nutrients (indicating that they may consume these nutrients), highlighting the potential role of these gut commensals for providing colonisation resistance against CRE in antibiotic-naïve faecal microbiota.

### Example 4—Carbapenem-Resistant Enterobacteriaceae can Grow Using Nutrients that were Elevated after Antibiotic Treatment of Faecal Microbiota (CRE)

Thus far the inventors have demonstrated that monosaccharides, disaccharides, amino acids, uracil, and succinate were elevated with antibiotic treatment of faecal cultures using antibiotics that are known to promote the intestinal colonisation of CRE (FIG. 2). Their next aim was to demonstrate that these nutrients support CRE growth. The growth of carbapenem-resistant E. coli, K. pneumoniae, and E. cloacae were measured in a mixture of these nutrients supplemented into M9 minimal medium (lacking any other carbon or nitrogen sources) and changes in OD were measured over time. Recent studies have suggested that antibiotic-mediated disruption of the gut microbiota promotes oxygenation of the gut, allowing Enterobacteriaceae to grow better on available carbon sources. Therefore, the inventors investigated the growth of E. coli, K. pneumoniae, and E. cloacae under both anaerobic and aerobic conditions.

The inventors showed that CRE growth was supported by the nutrient mixture under both anaerobic and aerobic conditions, when these nutrients were acting as the sole carbon and nitrogen sources (FIG. 5). However, for each CRE isolate growth was significantly higher under aerobic conditions compared to anaerobic conditions. Therefore, CRE can grow on nutrients that are elevated with antibiotics, and oxygenation of the gut with antibiotics has the potential to promote higher levels of CRE growth on these nutrients.

### Example 5—Carbapenem-Resistant Enterobacteriaceae can Use Monosaccharides, Disaccharides, and Amino Acids as Carbon Sources to Support their Growth (CRE)

Above the inventors demonstrated that a mixture of monosaccharides, disaccharides, amino acids, uracil, and succinate were able to support CRE growth in a minimal medium (FIG. 5). However, it was not clear which specific nutrients were utilised by each CRE isolate. Therefore, they next tested which individual nutrients could act as sole carbon sources to support CRE growth. The carbon utilisation abilities of E. coli, K. pneumoniae, and E. cloacae were demonstrated by adding a single carbon source into M9 minimal medium (containing ammonium chloride as the nitrogen source) and monitoring changes in OD over time. These experiments were performed under anaerobic and aerobic conditions.

When grown anaerobically, E. coli, K. pneumoniae, and E. cloacae could use most monosaccharides and disaccharides as carbon sources to support their growth (FIG. 6), with some carbon sources supporting higher levels of growth than others (FIG. 7). E. coli could use arabinose, fructose, galactose, glucose, mannose, xylose, N-acetylglucosamine, and trehalose. K. pneumoniae could use arabinose, fructose, galactose, glucose, mannose, xylose, N-acetylglucosamine, maltose, sucrose, and trehalose. E. cloacae could use arabinose, fructose, galactose, glucose, mannose, ribose, xylose, N-acetylglucosamine, maltose, sucrose, and trehalose. The inventors also showed that the CRE isolates could use some more complex substrates as carbon sources to support their growth (FIG. 8).

When grown aerobically, E. coli, K. pneumoniae, and E. cloacae could use monosaccharides, disaccharides, and some amino acids as carbon sources to support their growth (FIG. 6), again with some carbon sources supporting higher levels of growth than others (FIG. 7). Under aerobic conditions, E. coli could use the same carbon sources that it could use under anaerobic conditions but was also able to grow on ribose, maltose, sucrose, and alanine. K. pneumoniae could also use the same carbon sources that it could use under anaerobic conditions but was also able to grow on ribose, alanine, glutamate, and proline. E. cloacae could also use the same carbon sources that it could use under anaerobic conditions but was also able to grow on fucose, alanine, and glutamate.

Overall, E. coli, K. pneumoniae, and E. cloacae were able to utilise individual nutrients as carbon sources to support their growth. However, each isolate was able to grow to higher ODs when grown aerobically vs anaerobically. Moreover, each isolate was also able to grow on a larger number of carbon sources aerobically vs anaerobically.

### Example 6—Carbapenem-Resistant Enterobacteriaceae can Use Amino Acids, N-Acetylglucosamine, and Uracil as Nitrogen Sources to Support their Growth (CRE)

Above the inventors showed that most amino acids and uracil were not used as carbon sources by CRE. However, amino acids and uracil may instead act as nitrogen sources to support CRE growth. Therefore, they next tested whether amino acids, uracil, or N-acetylglucosamine could act as sole nitrogen sources to support CRE growth. The nitrogen utilisation abilities of E. coli, K. pneumoniae, and E. cloacae were demonstrated by adding a single nitrogen source into M9 minimal medium (containing glucose as the carbon source) and monitoring changes in OD over time. These experiments were performed under anaerobic and aerobic conditions.

When grown anaerobically, E. coli, K. pneumoniae, and E. cloacae could use some amino acids and N-acetylglucosamine as nitrogen sources to support their growth (FIG. 9), with some nitrogen sources supporting higher levels of growth than others (FIG. 10). E. coli could use arginine, aspartate, valine, and N-acetylglucosamine. K. pneumoniae and E. cloacae could use arginine, aspartate, and N-acetylglucosamine.

When grown aerobically, E. coli, K. pneumoniae, and E. cloacae could use most amino acids, N-acetylglucosamine, and uracil as nitrogen sources to support their growth (FIG. 9), again with some nitrogen sources supporting higher levels of growth than others (FIG. 10). Under aerobic conditions E. coli could use the same nitrogen sources that it could use under anaerobic conditions but was also able to grow on additional nitrogen sources: alanine, glutamate, glycine, isoleucine, leucine, lysine, methionine, proline, threonine, tryptophan, tyrosine, and uracil. Under aerobic conditions K. pneumoniae could use some of the nitrogen sources that it could use under anaerobic conditions (arginine, aspartate) but was not able to use N-acetylglucosamine. However, K. pneumoniae was also able to grow on additional nitrogen sources: alanine, glutamate, glycine, isoleucine, leucine, lysine, methionine, proline, and uracil. E. cloacae could also use the same nitrogen sources that it could use under anaerobic conditions but was also able to grow on additional nitrogen sources: alanine, glutamate, glycine, isoleucine, leucine, lysine, methionine, phenylalanine, proline, threonine, and uracil.

Overall, E. coli, K. pneumoniae, and E. cloacae were able to utilise individual nutrients as nitrogen sources to support their growth. Again, each isolate was able to grow to higher ODs when grown aerobically vs anaerobically. Moreover, each isolate was also able to grow on a larger number of nitrogen sources aerobically vs anaerobically.

### Example 7—Nutrient Preference and Utilisation Rate Varies Between Different CRE Isolates and Under Aerobic and Anaerobic Conditions (CRE)

Above the inventors demonstrated that CRE can use monosaccharides, disaccharides, and some amino acids as carbon sources and amino acids, N-acetylglucosamine, and uracil as nitrogen sources to support their growth. However, carbon utilisation assays were carried out using a single nitrogen source (ammonium chloride) and nitrogen utilisation assays were carried out using a single carbon source (glucose). As demonstrated in FIG. 2, there are several carbon and nitrogen sources that are available to support CRE growth following antibiotic treatment, and it is not clear whether CRE have preferences for some carbon or nitrogen sources over others. It is also not clear whether the presence of some carbon or nitrogen sources will influence the utilisation of other carbon or nitrogen sources. Therefore, the nutrient utilisation abilities of E. coli, K. pneumoniae, and E. cloacae were measured by adding a mixture of these nutrients into the M9 minimal medium (lacking any other carbon or nitrogen sources) and monitoring changes in nutrient concentration over time by 1H-NMR spectroscopy. Again, these experiments were performed under anaerobic and aerobic conditions.

In terms of sugar utilisation, E. coli, K. pneumoniae, and E. cloacae were able to utilise nearly all the monosaccharides and disaccharides tested (FIG. 11). When grown anaerobically, E. coli and E. cloacae had high utilisation of all monosaccharides and disaccharides. K. pneumoniae had high utilisation of all monosaccharides and disaccharides except for fucose, which was not utilised. When grown aerobically, E. coli had high utilisation of all monosaccharides and disaccharides except for sucrose, which had moderate utilisation. K. pneumoniae had high utilisation of all monosaccharides and disaccharides except for fucose, which was not utilised. E. cloacae had high utilisation of all monosaccharides and disaccharides.

In terms of amino acid utilisation, E. coli, K. pneumoniae, and E. cloacae showed variable utilisation patterns. Under anaerobic conditions, E. coli had high utilisation of aspartate and lysine, moderate utilisation of tryptophan, and low utilisation of the other amino acids (except for alanine or proline, which were not utilised). K. pneumoniae had high utilisation of aspartate and lysine, and low utilisation of the other amino acids (except for alanine, proline, and tryptophan, which were not utilised). E. cloacae had high utilisation of aspartate, threonine, and arginine, and low utilisation of the other amino acids (except for alanine, proline, tryptophan, and tyrosine, which were not utilised). E. cloacae also produced low levels of lysine. Under aerobic conditions, E. coli had high utilisation of aspartate and tryptophan, moderate utilisation of glutamate, lysine, and threonine, and low utilisation of the other amino acids (except for proline, which was not utilised). K. pneumoniae had high utilisation of aspartate, lysine, and threonine, moderate utilisation of glutamate, and low utilisation of the other amino acids (except for tryptophan, which was not utilised). E. cloacae had high utilisation of aspartate, threonine, and arginine, moderate utilisation of glutamate, and low utilisation of the other amino acids (except for proline, tryptophan, and tyrosine, which were not utilised). Again, E. cloacae also produced low levels of lysine.

Uracil utilisation also varied by isolate and under anaerobic and aerobic conditions. Under anaerobic conditions E. coli had moderate utilisation of uracil, and K. pneumoniae and E. cloacae did not utilise uracil. Under aerobic conditions E. coli had moderate utilisation of uracil, K. pneumoniae had low utilisation of uracil, and E. cloacae did not use uracil.

The rate at which nutrients were utilised varied depending on the CRE isolate and the conditions under which the CRE isolates were cultured (FIG. 12). The inventors found that some nutrients were used within different utilisation windows (0-4 hours, 0-8 hours, 4-8 hours, 4-24 hours and 0-24 hours). The nutrients utilised within these windows varied between the different CRE isolates, indicating that each CRE isolate had its own ordered preference of nutrients. Moreover, aerobic cultures utilised most nutrients more rapidly than anaerobic cultures.

Succinate was produced by all CRE isolates under both anaerobic and aerobic conditions. Other metabolites produced included acetate, ethanol, and formate (FIG. 13).

Overall, E. coli, K. pneumoniae, and E. cloacae were able to utilise most monosaccharides and disaccharides under both anaerobic and aerobic conditions. Amino acid and uracil utilisation varied between different CRE isolates and for the same CRE isolate grown under anaerobic and aerobic conditions. The rate at which the nutrients were utilised also varied by CRE isolate and differed for CRE isolates grown under anaerobic or aerobic conditions. Generally, there was more rapid nutrient utilisation under aerobic conditions, potentially linked to the higher growth rate of CRE isolates under aerobic conditions (see FIG. 5).

### Example 8—Carbapenem-Resistant Enterobacteriaceae Growth is Inhibited by Microbial Metabolites that were Decreased in Antibiotic-Treated Faecal Microbiota (CRE)

An increase in nutrient availability may be linked to reduced metabolite production. The inventors demonstrated that microbial metabolites were decreased in antibiotic-treated faecal cultures (FIG. 2). They next tested whether these metabolites could inhibit CRE growth.

First, the inventors determined whether a mixture of these metabolites (formate, acetate, propionate, butyrate, valerate, isobutyrate, isovalerate, lactate, 5-aminovalerate, and ethanol) were able to inhibit CRE growth. LB was supplemented with a mixture of these metabolites at concentrations mimicking their average concentrations measured in healthy human faeces. As SCFAs have variable inhibition abilities at different pH, the metabolite mixture was tested at three pH conditions mimicking the pH found in the large intestine: 6.0, 6.5, and 7.0. OD was measured after incubation for 16 hours under anaerobic conditions. The inventors showed that the metabolite mixture inhibited the growth of E. coli, K. pneumoniae, and E. cloacae at pH 6.0, 6.5, and 7.0 (FIG. 14). However, inhibition was higher at pH 6.0 and 6.5 compared to pH 7.0.

Next, they measured CRE growth in the presence of each individual metabolite at three different concentrations: the average, minimum, and maximum concentrations measured in healthy faeces (see Table 1). These experiments were performed at pH 6.5 as this is representative of the pH found in the large intestine, and this pH also showed strong inhibition in the mixed metabolite experiment.

The inventors showed that E. coli, K. pneumoniae, and E. cloacae growth was inhibited by specific microbial metabolites (FIG. 15). E. coli was inhibited by acetate at low, average, and high concentrations, by propionate, valerate, and ethanol at average and high concentrations, and by butyrate at high concentrations. K. pneumoniae was inhibited by butyrate at low, average, and high concentrations, and by acetate, propionate, valerate, isobutyrate, and isovalerate at average and high concentrations. E. cloacae was inhibited by butyrate and valerate at low, average, and high concentrations, by acetate, propionate, isobutyrate, and isovalerate at average and high concentrations, and by ethanol at the high concentration.

Overall, the inventors demonstrated that specific microbial metabolites that were decreased following antibiotic treatment of faecal microbiota were inhibitory towards CRE isolates. This suggests that the significant decrease of these metabolites following antibiotics is linked to the loss of colonisation resistance against CRE.

### Example 9—Antibiotic Treatment Decreased the Abundance of Important Bacterial Families in Faecal Microbiota (VRE)

The inventors used antibiotics to study how the loss or reduction of gut commensals from healthy gut microbiota impacted nutrient availability and metabolite production. The inventors performed ex vivo faecal culture experiments to measure the effects of five broad-spectrum antibiotics (known to promote the intestinal colonisation with VRE) on faecal microbiota from 12 healthy donors. Antibiotics tested included metronidazole (MTZ), vancomycin (VAN), piperacillin/tazobactam (TZP), ceftriaxone (CRO), and clindamycin (CLI). For each experiment a faecal sample was inoculated into a distal gut growth medium supplemented with one of the five antibiotics or not supplemented with antibiotics (antibiotic-naïve control), and incubated anaerobically for 24 hours.

First, the inventors aimed to determine whether antibiotics that promote VRE intestinal colonisation resulted in the loss or reduction of similar bacterial taxa across the different antibiotics. The inventors measured changes in bacterial taxa in the faecal cultures in response to antibiotics using 16S rRNA gene sequencing (to measure bacterial composition) and 16S rRNA qPCR (to measure bacterial biomass). The inventors found that antibiotics decreased the abundance of several bacterial families, as shown in FIG. 16. These antibiotics decreased the abundance of Bifidobacteriaceae, Coriobacteriaceae, Bacteroidales, and other bacterial families belonging to the Firmicutes and Proteobacteria phyla. Therefore, antibiotic treatment resulted in a decrease in the abundance of important bacterial families in faecal cultures.

### Example 10—Antibiotic Treatment Increased the Concentration of Nutrients and Decreased the Concentration of Microbial Metabolites in Faecal Microbiota (VRE)

The nutrient-niche hypothesis predicts that antibiotic-mediated disruption of the gut microbiota leads to an increase in nutrient availability in the gut, promoting pathogen growth. However, the nutrients that may be impacted by antibiotics have not been described. Moreover, microbial metabolites can be inhibitory to pathogen growth, but antibiotics have also been shown to decrease the concentration of microbial metabolites. Therefore, the inventors next measured the effects of antibiotics on nutrient availability and metabolite production in the antibiotic-treated and antibiotic-naïve faecal cultures using ¹H-NMR spectroscopy.

Antibiotic treatment of faecal cultures increased the concentration of several nutrients, including monosaccharides, disaccharides, and amino acids (FIG. 17). Monosaccharides that increased with antibiotics included 6-carbon sugars (glucose, fructose, galactose, mannose, fucose), 5-carbon sugars (arabinose, ribose, xylose) and N-acetylglucosamine. Disaccharides that increased with antibiotics included maltose, sucrose, and trehalose. Amino acids that increased with antibiotics included alanine, arginine, aspartate, glutamate, glycine, isoleucine, leucine, lysine, methionine, phenylalanine, proline, threonine, tryptophan, tyrosine, and valine. Antibiotics also increased the concentration of uracil (a pyrimidine) and succinate (a carboxylic acid). Therefore, antibiotic treatment of a faecal microbiota resulted in an increase in the amount of nutrients that are available to support the growth of VRE.

Antibiotics also decreased the concentration of metabolites produced by the gut microbiota, including short chain fatty acids (SCFAs), branched chain fatty acids (BCFAs), carboxylic acids, and ethanol (FIG. 17). Short chain fatty acids that decreased included formate, acetate, propionate, butyrate and valerate. Branched chain fatty acids that decreased with antibiotics included isobutyrate. Carboxylic acids that decreased with antibiotics included lactate and 5-aminovalerate. Therefore, antibiotic treatment resulted in a decrease in the concentration of microbial metabolites which may be important for maintaining colonisation resistance against VRE.

### Example 11—Vancomycin-Resistant Enterococcus can Use Monosaccharides, Disaccharides, and Amino Acids as Carbon Sources to Support their Growth (VRE)

Next the inventors tested which individual nutrients could act as sole carbon sources to support VRE growth. The carbon utilisation abilities of E. faecalis and E. faecium were demonstrated by adding a single carbon source into a minimal medium and monitoring changes in OD over time. These experiments were performed under aerobic conditions.

E. faecalis and E. faecium could use most monosaccharides and disaccharides as carbon sources to support their growth (FIG. 18), with some carbon sources supporting higher levels of growth than others. E. faecalis could use fructose, galactose, glucose, mannose, ribose, N-acetylglucosamine, maltose, sucrose, and trehalose. E. faecium could use arabinose, fructose, galactose, glucose, mannose, ribose, N-acetylglucosamine, maltose, trehalose, and tryptophan.

Overall, E. faecalis and E. faecium were able to utilise individual nutrients as carbon sources to support their growth.

### Example 12—Vancomycin-Resistant Enterococcus can Use Amino Acids as Nitrogen Sources to Support their Growth (VRE)

Above the inventors showed that most amino acids and uracil were not used as carbon sources by VRE. However, amino acids and uracil may instead act as nitrogen sources to support VRE growth. Therefore, they next tested whether amino acids, uracil, or N-acetylglucosamine could act as sole nitrogen sources to support VRE growth. The nitrogen utilisation abilities of E. faecalis and E. faecium were demonstrated by adding a mixture nitrogen source into a minimal medium, then leaving one of the nitrogen sources out of the mixture to determine its impact growth (changes in OD over time). These experiments were performed under aerobic conditions.

E. faecalis and E. faecium could use some amino acids as nitrogen sources to support their growth (FIG. 19), with some nitrogen sources supporting higher levels of growth than others. E. faecalis could use arginine, aspartate, cysteine, glutamate, glycine, histidine, isoleucine, leucine, lysine, methionine, serine, tryptophan, and valine. E. faecium could use arginine, glutamate, glycine, histidine, isoleucine, leucine, lysine, methionine, phenylalanine, serine, threonine, tryptophan, and valine.

Overall, E. faecalis and E. faecium were able to utilise amino acids as nitrogen sources to support their growth.

### Example 13—Carbapenem-Resistant E. coli Growth was More Inhibited in the Presence of a Combination of a Metabolite Mixture+Commensal Bacteria than in the Presence of Either the Metabolite Mixture Alone or the Commensal Mixture Alone

Referring to FIG. 20, the inventors demonstrate that a combination of commensal gut microorganisms and inhibitory metabolites suppresses the growth of carbapenem-resistant E. coli. Furthermore, the combination of commensal gut microorganisms and inhibitory metabolites was more suppressive to the growth of CRE than either mixture in isolation. These data provide compelling evidence that a combination of live commensal microorganisms and metabolites would be more effective in restricting CRE growth.

### DISCUSSION

In this study, the inventors demonstrated that antibiotic-mediated disruption of the gut microbiota (using antibiotics that promote CRE or VRE intestinal colonisation) led to the development of a nutrient-enriched and metabolite-depleted niche that was favourable for CRE or VRE growth. They showed that CRE and VRE can use these elevated nutrients as carbon and nitrogen sources to support their growth, where CRE and VRE show an order of preference for specific nutrients. CRE and VRE growth was higher on these nutrients in an oxygenated environment. This supports the hypothesis that oxygenation of the gut following antibiotic treatment allows CRE and VRE to grow better on available nutrients. The inventors also showed that specific microbial metabolites that were decreased with antibiotics were able to inhibit CRE growth.

The nutrient-niche hypothesis predicts that expansion of pathogens such as Enterobacteriaceae in an antibiotic-disturbed gut microbiota is due to an increase in the availability of nutrients, where competition for these nutrients would normally restrict their growth. This highlights the importance of measuring changes in nutrient availability following antibiotics and the importance of measuring nutrient utilisation in pathogens. The growth medium utilised in this study contained a mixture of complex carbohydrates, proteins, and mucin. The monosaccharides, disaccharides, and amino acids of interest in this study were derived from the breakdown of these more complex substrates, and therefore underlines the important role of microbial cross-feeding in the nutrient-enriched niche that was generated following antibiotic treatment. Glucose, maltose, and trehalose can be derived from starch. Fructose, glucose, and sucrose can be derived from inulin. Glucose, arabinose, galactose, xylose, mannose, and fucose can be derived from pectin. Xylose and arabinose can be derived from xylan. Arabinose and galactose can be derived from arabinogalactan. N-acetylglucosamine, galactose, fucose, and amino acids can be derived from mucin. Amino acid can also be derived from casein and peptone. Amino acids, ribose, and uracil can be derived from yeast extract. This means that CRE and VRE have a growth advantage in antibiotic-perturbed gut microbiota by acting as secondary fermenters of these nutrients.

The inventors demonstrated that antibiotics (that promote susceptibility to CRE or VRE) increased the availability of nutrients that can support CRE or VRE expansion within the gut. In this study, the inventors found an enrichment of nutrients associated with polysaccharides, mucin, and proteins, indicating a disruption of secondary fermentation in the gut.

The inventors showed that healthy faeces treated with antibiotics that promote CRE or VRE intestinal colonisation resulted in a decrease of several bacterial families, in particular Bifidobacteriaceae, Bacteroidales, and Coriobacteriaceae. This highlights the possible role of Bifidobacterium and Bacteroides in decolonising CRE-positive patients following FMT.

Bacteroides are important primary degraders and Bifidobacterium are important secondary degraders in the gut. In cross-feeding interactions, primary degraders break down complex carbohydrates, proteins, and mucin which results in the extracellular release of breakdown products (oligosaccharides, disaccharides, monosaccharides, peptides, and amino acids) which are then fermented by secondary degraders. Bifidobacterium have been shown to use most of the monosaccharides and disaccharides tested in this study. Bifidobacterium have weak protease activity, and amino acid utilisation has not been well characterised. However, Bifidobacterium have been shown to utilise alanine, aspartate, glutamate, and threonine as sole carbon sources. Bacteroides have been shown to utilise all the monosaccharides tested in this study. Bacteroides have proteolytic activity and can utilise both proteins and amino acids. Collinsella (Coriobacteriaceae) can utilise most monosaccharides and disaccharides tested in this study and are known to metabolise amino acids.

The inventors showed that monosaccharides and disaccharides released from polysaccharides and mucin are important carbon sources that promote the expansion of CRE and VRE in an antibiotic-disrupted gut.

The inventors also showed that CRE and VRE were able to utilise more nutrients when supplied as a mixture compared to their utilisation as the sole carbon or nitrogen source. These results highlight the importance of studying nutrient utilisation as a mixture of nutrients in addition to their utilisation as sole nutrient sources.

It has been hypothesised that facultatively anaerobic pathogens such and CRE and VRE can utilise oxygen (elevated after antibiotic treatment) to drive their expansion in the gut. In this study, the inventors showed that the growth of CRE and VRE on nutrients that were enriched with antibiotics was significantly higher under aerobic conditions compared to anaerobic conditions. Overall, this work supports the hypothesis that CRE and VRE growth is enhanced on carbon and nitrogen sources that become available due to antibiotic treatment under oxygenated conditions.

The metabolism of monosaccharides and disaccharides produces short chain fatty acids (SCFAs) and the metabolism of amino acids produces short chain fatty acids (SCFAs) and branched chain fatty acids (BCFAs). The inventors demonstrated that antibiotic treatment of the faecal microbiota results in the disruption of nutrient metabolism and the development of a metabolite depleted environment. In addition to acetate, propionate, and butyrate, the inventors also showed that formate, valerate, isobutyrate, isovalerate, lactate, 5-aminovalerate, and ethanol were decreased by antibiotics. The inventors showed that a mixture of these 10 metabolites was inhibitory against CRE at both acidic and neutral pH values representative of the human large intestine (pH 6.0, 6.5, 7.0). However, the metabolite mixture was more inhibitory at pH 6.0 and 6.5 compared to pH 7.0.

The inventors also demonstrated the inhibitory effects of each individual metabolite at a range of concentrations spanning those found in healthy human faeces. In addition to acetate, propionate, and butyrate, the inventors also demonstrated the inhibitory effects of valerate, isobutyrate, isovalerate, and ethanol. Differences in the concentration of these metabolites found in healthy faecal donors may contribute to the variable efficacy of FMT to decolonise CRE, where donors that produce high levels of inhibitory metabolites may be more effective than donors that produce lower levels.

Bacterial families that were decreased with antibiotics are known to produce these inhibitory metabolites. For example, Bifidobacterium are known to produce acetate, lactate, ethanol, and formate. Bacteroides are known to produce acetate, propionate, lactate, formate, ethanol, isobutyrate, and isovalerate. Collinsella can produce ethanol, formate, and lactate.

The inventors demonstrated that E. coli, K. pneumoniae, and E. cloacae are able to utilise some of these complex substrates to support their growth. Therefore, if the utilisation of these nutrients by primary fermenters (such as Bacteroides) decreases, then this would provide an additional growth advantage for CRE. Understanding how antibiotics disrupt gut microbiota-mediated colonisation resistance against CRE and VRE is critical for the design of microbiome therapeutics that aim to prevent or treat CRE or VRE intestinal colonisation. Microbiome therapeutics could reduce the risk of patients developing invasive diseases, reduce the recurrence of invasive diseases in chronically colonised patients, and reduce the spread of CRE or VRE to susceptible patients. As shown here, antibiotics create a niche that is favourable for CRE or VRE expansion in the gut by enriching for nutrients that support CRE or VRE growth and by depleting metabolites that are inhibitory towards CRE growth. These data support the rational design of microbiome therapeutics that reverse the effects of antibiotics by restoring gut commensal bacteria that can utilise nutrients that were enriched with antibiotics and convert these nutrients into inhibitory metabolites that were depleted with antibiotics.

As discussed, the human gut microbiota restricts the growth of pathogenic bacteria through multiple methods referred to as colonisation resistance⁷, and antibiotic treatment disrupts the native gut microbiota population which results in a reduction in microbial abundance and diversity, accompanied by a decrease in SCFA concentration^{1,4-6}. This loss of colonisation resistance leads to an increased susceptibility to infection from multi-drug resistant microorganisms. Therapies which aim to restore the colonisation resistance lost following antibiotic treatments can represent a novel therapeutic avenue to promote the decolonisation of MDR microorganisms. From the data presented in FIG. 20, the inventors demonstrated that a combination of commensal gut microorganisms and inhibitory metabolites suppresses the growth of carbapenem-resistant E. coli to a greater extent than either mixture alone. These data provide compelling evidence that a combination of live commensal microorganisms and inhibitory metabolites would be more effective in restricting CRE or VRE growth.

### CONCLUSIONS

The intestine is the primary colonisation site for carbapenem-resistant Enterobacteriaceae (CRE) and vancomycin-resistant Enterococcus (VRE) and serves as a reservoir for CRE/VRE responsible for invasive infections (e.g. bloodstream infections). Antibiotics disrupt microbiome-mediated colonisation resistance and promotes the expansion and domination of CRE and VRE within the intestine. Understanding how antibiotics disrupt colonisation resistance against CRE and VRE would enable the design of novel microbiome therapeutics to prevent or treat CRE or VRE intestinal colonisation, which would lead to decreased development of subsequent difficult-to-treat invasive infections.

The inventors have demonstrated that antibiotics (that promote susceptibility to CRE or VRE intestinal colonisation) disrupt nutrient metabolism in a manner that is favourable for CRE/VRE growth. Antibiotic-disrupted microbiota had an enrichment of nutrients that support CRE/VRE growth, coupled with a corresponding decrease in microbial metabolites that inhibit CRE/VRE growth. CRE or VRE can use these elevated nutrients as both carbon and nitrogen sources, where CRE/VRE show an order of preference for specific nutrients. CRE or VRE growth on these nutrients was higher in an oxygenated environment, suggesting that oxygenation of the gut (which has been associated with antibiotics) gives CRE/VRE a competitive advantage against anaerobic gut commensals. The inventors also demonstrated that microbial metabolites that were decreased with antibiotics were able to inhibit CRE or VRE growth. Overall, these findings support the design of microbiome therapeutics to prevent or treat CRE or VRE intestinal colonisation by restoring nutrient competition and the production of inhibitory metabolites. Furthermore, the inventors have surprisingly demonstrated that a combination of commensal gut microorganisms and inhibitory metabolites suppresses the growth of carbapenem-resistant E. coli, and that the combination of commensal gut microorganisms and inhibitory metabolites was more suppressive to the growth of CRE than either mixture in isolation, thus providing compelling evidence that a combination of live commensal microorganisms and metabolites would be more effective in restricting CRE growth.

Thus, the inventors have developed a novel microbiome-based therapeutic which comprises:
(a) a synthetic microbial consortium selected from Bifidobacteriaceae (e.g. Bifidobacterium), Bacteroidales (e.g. Bacteroides) and Coriobacteriaceae (e.g. Collinsella); and
(b) one or more inhibitory metabolites selected from formate, acetate, propionate, butyrate, isobutyrate, valerate, isovalerate, 5-aminovalerate, lactate and ethanol.

The inhibitory metabolites cause an initial reduction in CRE (or VRE) growth. The synthetic microbial consortium then can colonise the intestine, and outcompete the CRE or VRE for nutrients, and thereby inhibit CRE or VRE growth by producing further inhibitory metabolites, resulting in a long-term reduction in CRE or VRE growth in the gut.

### REFERENCES

1. Yip, A. Y. G. et al. Antibiotics promote intestinal growth of carbapenem-resistant Enterobacteriaceae by enriching nutrients and depleting microbial metabolites. 2023.03.25.533086 Preprint at https://doi.org/10.1101/2023.03.25.533086 (2023).
2. Shelton, C. D. et a/. Salmonella enterica serovar Typhimurium uses anaerobic respiration to overcome propionate-mediated colonization resistance. Cell Rep. 38, 110180 (2022).
3. Payne, A. N., Zihler, A., Chassard, C. & Lacroix, C. Advances and perspectives in in vitro human gut fermentation modeling. Trends Biotechnol. 30, 17-25 (2012).
4. Lange, K., Buerger, M., Stallmach, A. & Bruns, T. Effects of Antibiotics on Gut Microbiota. Dig. Dis. 34, 260-268 (2016).
5. Dethlefsen, L., Huse, S., Sogin, M. L. & Relman, D. A. The pervasive effects of an antibiotic on the human gut microbiota, as revealed by deep 16S rRNA sequencing. PLoS Biol. 6, e280 (2008).
6. Theriot, C. M. et al. Antibiotic-induced shifts in the mouse gut microbiome and metabolome increase susceptibility to Clostridium difficile infection. Nat. Commun. 5, 3114 (2014).
7. Horrocks, V., King, O. G., Yip, A. Y. G., Marques, I. M. & McDonald, J. A. K. Role of the gut microbiota in nutrient competition and protection against intestinal pathogen colonization. Microbiology 169, 001377 (2023).

1. (i) One or more microorganism selected from a group of microorganisms consisting of: Bifidobacteriaceae; Bacteroidales; and Coriobacteriaceae; and/or (ii) one or more compound comprising a short chain alcohol or a C₁-C₁₀ carboxylate or carboxylic acid, wherein the carboxylate or carboxylic acid is optionally substituted with OH and/or NH₂, or a pharmaceutically acceptable salt, solvate, tautomeric form or polymorphic form thereof, for use in treating, preventing or ameliorating an infection or intestinal colonisation of carbapenem-resistant Enterobacteriaceae (CRE) and/or vancomycin-resistant Enterococcus (VRE).

2. The one or more microorganism and/or the one or more compound, for use according to claim 1, wherein the one or more microorganism comprises a microorganism selected from Bifidobacteriaceae, optionally wherein the one or more Bifidobacteriaceae microorganism is a Bifidobacterium selected from a group consisting of: Bifidobacterium pseudocatenulatum, Bifidobacterium kashiwanohense, Bifidobacterium catenulatum, Bifidobacterium bifidum, Bifidobacterium longum, Bifidobacetrium merycicum, Bifidobacterium angulatum, Bifidobacterium thermacidophilum, Bifidobacterium thermophilum, and Bifidobacterium adolescentis.

3. The one or more microorganism and/or the one or more compound, for use according to either claim 1 or claim 2, wherein a plurality of Bifidobacteriaceae microorganisms are used to treat, prevent or ameliorate an infection or intestinal colonisation of carbapenem-resistant Enterobacteriaceae (CRE) and/or vancomycin-resistant Enterococcus (VRE), wherein at least 1×10⁶, at least 1×10⁷, at least 1×10⁸, at least 1×10⁹, or at least 1×10¹⁰ Bifidobacteriaceae microorganisms or CFU are used.

4. The one or more microorganism and/or the one or more compound, for use according to any preceding claim, wherein the one or more microorganism comprises a microorganism selected from Bacteroidales, optionally wherein the one or more Bacteroidales microorganism is a Bacteroides selected from a group consisting of: Bacteroides pectinophilus, Bacteroides plebius, Bacteroides xylanisolvens, Bacteroides ovatus, Bacteroides intestinalis, Bacteroides eggerthi, Bacteroides coprophilus, Bacteroides dorei, Bacteroides coprocola, Bacteroides vulgatus, Bacteroides uniformis, Bacteroides massiliensis, Bacteroides fragilis, Bacteroides stercoris, Bacteroides thetaiotaomicron, Bacteroides caccae, Barnesiella intestinihominis, Barnesiella propionica, Prevotella copri, Alistipes finegoldii, Alistipes indistinctus, Alistipes inops, Alistipes massiliensis, Alistipes onderdonkii, Alistipes putredinis, Alistipes shahii, Alistipes timonensis, Parabacteroides distasonis, and Parabacteroides merdae.

5. The one or more microorganism and/or the one or more compound, for use according to any preceding claim, wherein a plurality of Bacteroidales microorganisms are used to treat, prevent or ameliorate an infection or intestinal colonisation of carbapenem-resistant Enterobacteriaceae (CRE) and/or vancomycin-resistant Enterococcus (VRE), wherein at least 1×10⁶, at least 1×10⁷, at least 1×10⁸, at least 1×10⁹, or at least 1×10¹⁰ Bacteroidales microorganisms or CFU are used.

6. The one or more microorganism and/or the one or more compound, for use according to any preceding claim, wherein the one or more microorganism comprises a microorganism selected from Coriobacteriaceae, optionally wherein the one or more Coriobacteriaceae microorganism is a Collinsella selected from a group consisting of: Collinsella aerofaciens, Collinsella tanakaei, Collinsella acetigenes, Collinesella bouchesdurhonensis, Collinsella ihuae, Collinsella ihumii, Collinsella intestinalis, Collinsella massiliensis, Collinsella phocaeensis, Collinsella provencensis, and Collinsella stercoris.

7. The one or more microorganism and/or the one or more compound, for use according to any preceding claim, wherein a plurality of Coriobacteriaceae microorganisms are used to treat, prevent or ameliorate an infection or intestinal colonisation of carbapenem-resistant Enterobacteriaceae (CRE) and/or vancomycin-resistant Enterococcus (VRE), wherein at least 1×10⁶, at least 1×10⁷ or at least 1×10⁸, at least 1×10⁹, or at least 1×10¹⁰ Coriobacteriaceae microorganisms or CFU are used.

8. The one or more microorganism and/or the one or more compound, for use according to any preceding claim, wherein the one or more microorganism comprises at least two microorganisms selected from: Bifidobacteriaceae; Bacteroidales; and Coriobacteriaceae, optionally wherein the at least two microorganisms comprise Bifidobacteriaceae and Bacteroidales; or Bifidobacteriaceae and Coriobacteriaceae; or Bacteroidales and Coriobacteriaceae.

9. The one or more microorganism and/or the one or more compound, for use according to any preceding claim, wherein the one or more microorganism comprises all three microorganisms of Bifidobacteriaceae; Bacteroidales; and Coriobacteriaceae.

10. The one or more microorganism and/or the one or more compound, for use according to any preceding claim, wherein (a) the ratio of the amount of Bifidobacteriaceae:Coriobacteriaceae which is used is at least 1.5:1, at least 5:1, at least 10:1, at least 15:1, or no more than 18:1; and/or (b) wherein the ratio of the amount of Bacteroidales:Coriobacteriaceae which is used is at least 3:1, at least 10:1, at least 20:1, at least 30:1, or no more than 36:1.

11. The one or more microorganism and/or the one or more compound, for use according to any preceding claim, wherein the one or more microorganism is administered orally to a subject in need of treatment or prophylaxis.

12. The one or more microorganism and/or the one or more compound, for use according to any preceding claim, wherein the one or more microorganism is formulated into a pill, capsule or a liquid suspension.

13. The one or more microorganism and/or the one or more compound, for use according to any preceding claim, wherein the short chain alcohol is a C₁-C₇ alcohol, a C₁-C₅ alcohol, or a C₁-C₃ alcohol.

14. The one or more microorganism and/or the one or more compound, for use according to any preceding claim, wherein the alcohol is ethanol.

15. The one or more microorganism and/or the one or more compound, for use according to any preceding claim, wherein the one or more compound comprises a C₁-C₁₀ carboxylate or carboxylic acid.

16. The one or more microorganism and/or the one or more compound, for use according to any preceding claim, wherein the carboxylate or carboxylic acid is a straight or branched chain carboxylate or carboxylic acid.

17. The one or more microorganism and/or the one or more compound, for use according to any preceding claim, wherein the carboxylate or carboxylic acid is substituted with an OH group.

18. The one or more microorganism and/or the one or more compound, for use according to any preceding claim, wherein the carboxylate or carboxylic acid is substituted with an NH₂ group.

19. The one or more microorganism and/or the one or more compound, for use according to any preceding claim, wherein the C₁-C₁₀ carboxylate or carboxylic acid is a short chain fatty acid or a long chain fatty acid.

20. The one or more microorganism and/or the one or more compound, for use according to any preceding claim, wherein the C₁-C₁₀ carboxylate or carboxylic acid is a C₁-C₇ carboxylate or carboxylic acid, or a C₁-C₆ carboxylate or carboxylic acid, or a C₁-C₅ carboxylate or carboxylic acid.

21. The one or more microorganism and/or the one or more compound, for use according to any preceding claim, wherein the carboxylate or carboxylic acid is a C₅ carboxylate or carboxylic acid, optionally wherein the C₅ carboxylate or carboxylic acid is valerate, isovalerate or 5-aminovalerate.

22. The one or more microorganism and/or the one or more compound, for use according to any preceding claim, wherein the carboxylate or carboxylic acid is a C₄ carboxylate or carboxylic acid, optionally wherein the C₄ carboxylate or carboxylic acid is butyrate or isobutyrate.

23. The one or more microorganism and/or the one or more compound, for use according to any preceding claim, wherein the carboxylate or carboxylic acid is a C₃ carboxylate or carboxylic acid, optionally wherein the C₃ carboxylate or carboxylic acid is propionate or lactate.

24. The one or more microorganism and/or the one or more compound, for use according to any preceding claim, wherein the carboxylate or carboxylic acid is a C₂ carboxylate or carboxylic acid, optionally wherein the C₂ carboxylate or carboxylic acid is acetate.

25. The one or more microorganism and/or the one or more compound, for use according to any preceding claim, wherein the carboxylate or carboxylic acid is a C₁ carboxylate or carboxylic acid, optionally wherein the C₁ carboxylate or carboxylic acid is formate.

26. The one or more microorganism and/or the one or more compound, for use according to any preceding claim, wherein the carboxylate or carboxylic acid is formate or formic acid, acetate or acetic acid, propionate or propionic acid, butyrate or butyric acid, isobutyrate or isobutyric acid, valerate or valeric acid, isovalerate or isovaleric acid, 5-aminovalerate or aminovaleric acid, or lactate or lactic acid.

27. The one or more microorganism and/or the one or more compound, for use according to any preceding claim, wherein at least one, two or three compounds selected from a group consisting of: (i) ethanol, (ii) formate or formic acid, (iii) acetate or acetic acid, (iv) propionate or propionic acid, (v) butyrate or butyric acid, (vi) isobutyrate or isobutyric acid, (vii) valerate or valeric acid, (viii) isovalerate or isovaleric acid, (ix) 5-aminovalerate or aminovaleric acid, and (x) lactate or lactic acid, is used to treat, prevent or ameliorate an infection or intestinal colonisation of CRE and/or VRE.

28. The one or more microorganism and/or the one or more compound, for use according to any preceding claim, wherein at least four, five or six compounds selected from a group consisting of: (i) ethanol, (ii) formate or formic acid, (iii) acetate or acetic acid, (iv) propionate or propionic acid, (v) butyrate or butyric acid, (vi) isobutyrate or isobutyric acid, (vii) valerate or valeric acid, (viii) isovalerate or isovaleric acid, (ix) 5-aminovalerate or aminovaleric acid, and (x) lactate or lactic acid, are used to treat, prevent or ameliorate an infection or intestinal colonisation of CRE and/or VRE.

29. The one or more microorganism and/or the one or more compound, for use according to any preceding claim, wherein at least seven, eight, nine or ten compounds selected from a group consisting of: (i) ethanol, (ii) formate or formic acid, (iii) acetate or acetic acid, (iv) propionate or propionic acid, (v) butyrate or butyric acid, (vi) isobutyrate or isobutyric acid, (vii) valerate or valeric acid, (viii) isovalerate or isovaleric acid, (ix) 5-aminovalerate or aminovaleric acid, and (x) lactate or lactic acid, are used to treat, prevent or ameliorate an infection or intestinal colonisation of CRE and/or VRE.

30. The one or more microorganism and/or the one or more compound, for use according to any preceding claim, wherein:
(i) between 0.5 mM and 180 mM of ethanol is administered, preferably about 10 mM;
(ii) between 0.05 mM and 0.3 mM of formate is administered, preferably about 0.10 mM;
(iii) between 10 mM and 200 mM of acetate is administered, preferably about 65 mM;
(iv) between 5 mM and 50 mM of propionate is administered, preferably about 15 mM;
(v) between 3 mM and 50 mM of butyrate is administered, preferably about 15 mM;
(vi) between 0.5 mM and 15 mM of valerate is administered, preferably about 3.5 mM;
(vii) between 0.5 mM and 6 mM of isobutyrate is administered, referably about 2 mM;
(viii) between 0.5 mM and 6 mM of isovalerate is administered, preferably about 2 mM;
(ix) between 0.25 mM and 4 mM of aminovalerate is administered, preferably about 1 mM; and/or
(x) between 0.25 mM and 2 mM of lactate is administered, preferably about 0.7 mM.

31. The one or more microorganism and/or the one or more compound, for use according to any preceding claim, wherein the compound is attached to, or conjugated with, a carrier molecule.

32. The one or more microorganism and/or the one or more compound, for use according to claim 31, wherein the carrier molecule comprises glycerol or a glycerol backbone.

33. The one or more microorganism and/or the one or more compound, for use according to any preceding claim, wherein the one or more microorganism, or the one or more compound, is used to treat, prevent or ameliorate colonisation of CRE and/or VRE in the gastrointestinal (GI) tract, preferably before they cause an invasive infection in the bloodstream.

34. The one or more microorganism and/or the one or more compound, for use according to any preceding claim, wherein an infection or intestinal colonisation of CRE and VRE is treated, prevented or ameliorated.

35. The one or more microorganism and/or the one or more compound, for use according to any preceding claim, wherein the carbapenem-resistant Enterobacteriaceae (CRE) is carbapenem-resistant Enterobacteriaceae, which is selected from a group consisting of carbapenem-resistant Escherichia, Klebsiella, Enterobacter, Citrobacter, Proteus, Serratia, and Salmonella, optionally wherein the CRE is selected from a group consisting of carbapenem-resistant Escherichia coli, Klebsiella pneumoniae, Klebsiella oxytoca, Klebsiella aerogenes, Enterobacter cloacae, Enterobacter cloacae complex (which encompasses Enterobacter asburiae, Enterobacter kobei, Enterobacter ludwigii, Enterobacter hormaechei subsp. oharae, subsp. hormaechei, and subsp. steigerwaltii, Enterobacter nimipressuralis, E. cloacae subsp. cloacae and subsp. dissolvens), Enterobacter gergoviae, Citrobacter freundii, Proteus mirabilis, Salmonella enterica, and Serratia marcescens.

36. The one or more microorganism and/or the one or more compound, for use according to any preceding claim, wherein the vancomycin-resistant Enterococcus (VRE) is vancomycin-resistant Enterococcaceae, preferably vancomycin-resistant Enterococcus selected from a group consisting of vancomycin-resistant Enterococcus faecalis, Enterococcus faecium, Enterococcus gallinarum, Enterococcus casseliflavus, Enterococcus durans, Enterococcus avium, and Enterococcus raffinosis.

37. The one or more microorganism and/or the one or more compound, for use according to any preceding claim, wherein either (i) one or more microorganism selected from a group of microorganisms consisting of: Bifidobacteriaceae; Bacteroidales; and Coriobacteriaceae; or (ii) one or more compound comprising a short chain alcohol or a C₁-C₁₀ carboxylate or carboxylic acid, wherein the carboxylate or carboxylic acid is optionally substituted with OH and/or NH₂, or a pharmaceutically acceptable salt, solvate, tautomeric form or polymorphic form thereof, is used to treat, prevent or ameliorate an infection or intestinal colonisation of CRE and/or VRE.

38. The one or more microorganism and/or the one or more compound, for use according to any preceding claim, wherein (i) one or more microorganism selected from a group of microorganisms consisting of: Bifidobacteriaceae; Bacteroidales; and Coriobacteriaceae; and (ii) one or more compound comprising a short chain alcohol or a C₁-C₁₀ carboxylate or carboxylic acid, wherein the carboxylate or carboxylic acid is optionally substituted with OH and/or NH₂, or a pharmaceutically acceptable salt, solvate, tautomeric form or polymorphic form thereof, are both used to treat, prevent or ameliorate an infection or intestinal colonisation of CRE and/or VRE.

39. The one or more microorganism and/or the one or more compound, for use according to claim 38, wherein the (i) one or more microorganism, and the (ii) one or more compound comprising a short chain alcohol or a C₁-C₁₀ carboxylate or carboxylic acid, are administered simultaneously or sequentially.
